(19) United States
(12) Patent Application Publication
(10) Pub. No.: US 2005/0020564 A1
(43) Pub. Date: Jan. 27, 2005

(54) Pyrazole-amides and sulfonamides as sodium channel modulators

(76) Inventors: Robert N. Atkinson, Raleigh, NC (US); Irene Drizin, Wadsworth, IL (US); Robert J. Gregg, Libertyville, IL (US); Michael F. Gross, Durham, NC (US); Michael E. Kort, Lake Bluff, IL (US); Lei Shi, Waukegan, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/838,087

(22) Filed: May 3, 2004

Related U.S. Application Data
(60) Provisional application No. 60/466,980, filed on May 1, 2003.

(51) Int. Cl. A61K 31/541; A61K 31/5377; A61K 31/496; A61K 31/454; A61K 31/416

(52) U.S. Cl. 514/210.2; 514/227.8; 514/235.5; 514/254.05; 514/326; 514/217.09; 514/406; 544/60; 544/140; 544/371; 546/211; 548/364.1

(57) ABSTRACT

Compounds of the present invention modulate PN3 in mammals and are useful in treating pain in mammals.

This application claim priority from U.S. Provisional Patent Application Ser. No. 60/466,980, filed May 1, 2003.

### TECHNICAL FIELD

The present invention relates to certain pyrazole-amide and pyrazole-sulfonamide compounds that modulate PN3 and are useful for treating neuropathic pain.

### BACKGROUND OF THE INVENTION

Sodium channel blockers are effective in the treatment of various disease states including neuropathic pain. Neuropathic pain can be described as pain associated with damage or permanent alteration of the peripheral or central nervous system. Clinical manifestations of neuropathic pain include a sensation of burning or electric shock, feelings of bodily distortion, allodynia, and hyperalgesia.

Sodium channel-blocking agents selectively suppress abnormal ectopic neural firing in injured peripheral and central neurons. Alterations in either the level of expression or distribution of sodium channels within an injured nerve, therefore, have a major influence on the pathophysiology of pain associated with this type of trauma.

Nav1.8 (also known as PN3) is a member of a family of voltage-gated sodium channels. PN3-nulled mutant mice exhibit a pronounced analgesia to mechanical noxious stimuli. Selective “knock down” of PN3 protein in the rat dorsal root ganglion with specific antisense oligodeoxynucleotides prevents hyperalgesia and allodynia caused by either chronic nerve or tissue injury. In both human and animal models of neuropathic pain, there is an increased expression of PN3 at the site of peripheral nerve injury.

Patients with neuropathic pain do not respond to non-steroidal anti-inflamatory drugs and resistance or insensitivity to opiates is common. Gabapentin is the market leading treatment for neuropathic pain; its mechanism of action for pain is unknown. As few as 30% of patients respond to gabapentin treatment.

In view of the limited number of agents presently available and the low levels of efficacy of the available agents, there is a pressing need for compounds that are potent, specific inhibitors of ion channels implicated in neuropathic pain. The present invention provides such compounds, methods of using them, and compositions that include the compounds.

### SUMMARY OF THE INVENTION

The present invention discloses pyrazole-amides and pyrazole-sulfonamides, a method for modulating PN3 in mammals using these compounds, a method for controlling pain in mammals, and pharmaceutical compositions including those compounds. More particularly, the present invention is directed to compounds of formula (I) 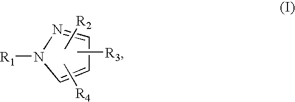
or a pharmaceutically acceptable salt, amide, ester, or prodrug thereof, wherein
R₁ is alkyl, aryl, arylalkyl, cycloalkyl, cycloalkylalkyl, haloalkyl, heterocycle, heterocyclealkyl, heteroaryl, or heteroarylalkyl;
R₂ and R₃ are independently hydrogen, alkenyl, alkoxy, alkoxyalkyl, alkoxycarbonyl, alkyl, alkylcarbonyl, alkylcarbonyloxy, alkylthio, alkynyl, aryl, arylalkyl, carboxy, cycloalkyl, cycloalkylalkyl, cyano, formyl, haloalkoxy, haloalkyl, halogen, hydroxy, hydroxyalkyl, mercapto, nitro, —NR_{A}R_{B}, or (NR_{A}R_{B})carbonyl;
R_{A} and R_{B} are independently hydrogen, alkyl, or alkylcarbonyl;
R₄ is 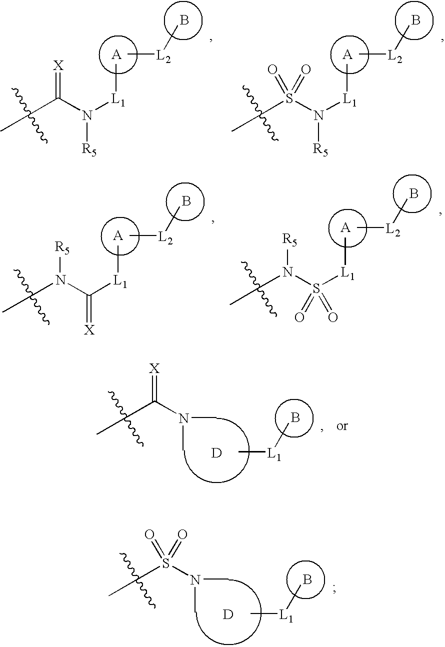
X is O or S;
R₅ is hydrogen, alkyl, alkylcarbonyl, alkylcarbonyloxy, or heterocyclealkyl;
L₁ is a bond or alkylene;
L₂ is a bond or alkylene;
A is aryl, cycloalkyl, heteroaryl, or heterocycle;
B is aryl, cycloalkyl, heteroaryl, or heterocycle;
D is heterocycle wherein the heterocycle is azetidinyl, azepanyl, aziridinyl, azocanyl, 1,1-dioxidothiomorpholinyl, morpholinyl, piperazinyl, piperidinyl, pyrrolidinyl, or thiomorpholinyl, wherein the heterocycle is optionally substituted with 1, 2, 3, or 4 substitutents independently selected from alkenyl, alkoxy, alkoxyalkyl, alkoxycarbonyl, alkoxysulfonyl, alkyl, alkylcarbonyl, alkylcarbonyloxy, alkylsulfonyl, alkynyl, carboxy, cyano, formyl, haloalkoxy, haloalkyl, halo, hydroxy, hydroxyalkyl, mercapto, —NR_{A}R_{B}, (NR_{A}R_{B})carbonyl, (NR_{A}R_{B})sulfonyl.

In another embodiment, the present invention discloses compounds of formula (II) that modulate PN3 in mammals and are useful for controlling pain in mammals, 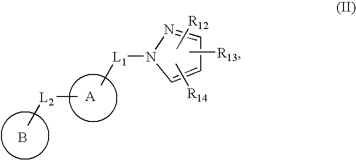
or a pharmaceutically acceptable salt, amide, ester, or prodrug thereof, wherein
R₁₂ and R₁₃ are independently hydrogen, alkenyl, alkoxy, alkoxyalkyl, alkoxycarbonyl, alkyl, alkylcarbonyl, alkylcarbonyloxy, alkylthio, alkynyl, aryl, arylalkyl, carboxy, cycloalkyl, cycloalkylalkyl, cyano, formyl, haloalkoxy, haloalkyl, halogen, hydroxy, hydroxyalkyl, mercapto, nitro, —NR_{A}R_{B}, or (NR_{A}R_{B})carbonyl;
R_{A} and R_{B} are independently hydrogen, alkyl, or alkylcarbonyl;
R₁₄ is 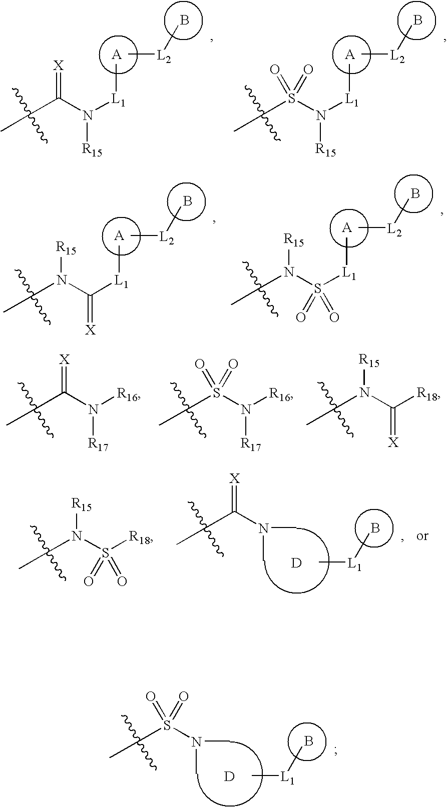
X is O or S;
R₁₅ is hydrogen, alkyl, alkylcarbonyl, alkylcarbonyloxy, or heterocyclealkyl;
R₁₆ and R₁₇ are independently hydrogen, alkenyl, alkoxy, alkyl, aryl, arylalkyl, cycloalkyl, cycloalkylalkyl, heteroaryl, heteroarylalkyl, heterocycle, or heterocyclealkyl;

R₁₈ is alkyl, alkenyl, aryl, arylalkyl, cycloalkyl, cycloalkylalkyl, heteroaryl, heteroarylalkyl, heterocycle, heterocyclealkyl, —NR_{A}R_{B}, or (NR_{A}R_{B})alkyl;
L₁ is a bond or alkylene;
L₂ is absent, a bond, or alkylene;
A is aryl, cycloalkyl, heteroaryl, or heterocycle;
B is absent, aryl, cycloalkyl, heteroaryl, or heterocycle;
D is heterocycle wherein the heterocycle is azetidinyl, azepanyl, aziridinyl, azocanyl, 1,1-dioxidothiomorpholinyl, morpholinyl, piperazinyl, piperidinyl, pyrrolidinyl, or thiomorpholinyl, wherein the heterocycle is optionally substituted with 1, 2, 3, or 4 substitutents independently selected from alkenyl, alkoxy, alkoxyalkyl, alkoxycarbonyl, alkyl, alkylcarbonyl, alkylcarbonyloxy, alkynyl, carboxy, cyano, formyl, haloalkoxy, haloalkyl, halo, hydroxy, hydroxyalkyl, mercapto, —NR_{A}R_{B}, or (NR_{A}R_{B})carbonyl.

In another embodiment, the present invention discloses compounds that modulate PN3 in mammals and are useful for controlling pain in mammals. These compounds include:
1-(3-chlorophenyl)-N-[3-(methylsulfonyl)phenyl]-5-(trifluoromethyl)-1H-pyrazole-4-carboxamide;
1-(3-methylphenyl)-N-[3-(methylsulfonyl)phenyl]-5-(trifluoromethyl)-1H-pyrazole-4-carboxamide;
1-(4-methylphenyl)-N-[3-(methylsulfonyl)phenyl]-5-(trifluoromethyl)-1H-pyrazole-4-carboxamide;
1-(2-methoxyphenyl)-5-(trifluoromethyl)-N-[3-(trifluoromethyl)benzyl]-1H-pyrazole-4-carboxamide;
N-[2-(4-chlorophenyl)ethyl]-1-(2-methoxyphenyl)-5-(trifluoromethyl)-1H-pyrazole-4-carboxamide;
1-cyclohexyl-5-(trifluoromethyl)-N-[3-(trifluoromethyl)benzyl]-1H-pyrazole-4-carboxamide;
1-cyclohexyl-N-[3-(methylsulfonyl)phenyl]-5-(trifluoromethyl)-1H-pyrazole-4-carboxamide;
N-[2-(4-chlorophenyl)ethyl]-1-(7-chloroquinolin-4-yl)-5-(trifluoromethyl)-1H-pyrazole-4-carboxamide;
1-(4-chlorophenyl)-5-methyl-N-[3-(trifluoromethyl)benzyl]-1H-pyrazole-4-carboxamide;
1-(4-chlorophenyl)-N-[2-(4-chlorophenyl)ethyl]-5-methyl-1H-pyrazole-3-carboxamide;
1-(4-chlorophenyl)-5-methyl-N-[3-(trifluoromethyl)benzyl]-1H-pyrazole-3-carboxamide;
1-(4-chlorophenyl)-5-methyl-N-[3-(methylsulfonyl)phenyl]-1H-pyrazole-3-carboxamide;
N-benzyl-1-(4-chlorophenyl)-5-hydroxy-1H-pyrazole-4-carboxamide;
1-(4-chlorophenyl)-5-hydroxy-N-[3-(methylsulfonyl)phenyl]-1H-pyrazole-4-carboxamide;
1-(4-chlorophenyl)-5-cyano-N-[3-(methylsulfonyl)phenyl]-1H-pyrazole-4-carboxamide;
1-(4-chlorophenyl)-N-[3-(methylsulfonyl)phenyl]-5-vinyl-1H-pyrazole-4-carboxamide;
1-(4-chlorophenyl)-N-(3,4-dichlorobenzyl)-5-vinyl-1H-pyrazole-4-carboxamide;
5-acetyl-1-(4-chlorophenyl)-N-[3-(methylsulfonyl)phenyl]-1H-pyrazole-4-carboxamide;
5-acetyl-1-(4-chlorophenyl)-N-(3,4-dichlorobenzyl)-1H-pyrazole-4-carboxamide;
-(4-chlorophenyl)-N-(2-methoxybenzyl)-5-methyl-1H-pyrazole-3-carboxamide;
and
1-(4-chlorophenyl)-5-methyl-N-(2-methylbenzyl)-1H-pyrazole-3-carboxamide; or a pharmaceutically acceptable salt, amide, ester, or prodrug thereof

### DETAILED DESCRIPTION OF THE PRESENT INVENTION

In one embodiment of the present invention, compounds of formula (I) are disclosed 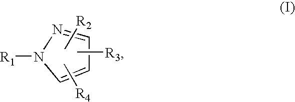
or a pharmaceutically acceptable salt, amide, ester, or prodrug thereof, wherein
R₁ is alkyl, aryl, arylalkyl, cycloalkyl, cycloalkylalkyl, haloalkyl, heterocycle, heterocyclealkyl, heteroaryl, or heteroarylalkyl;
R₂ and R₃ are independently hydrogen, alkenyl, alkoxy, alkoxyalkyl, alkoxycarbonyl, alkyl, alkylcarbonyl, alkylcarbonyloxy, alkylthio, alkynyl, aryl, arylalkyl, carboxy, cycloalkyl, cycloalkylalkyl, cyano, formyl, haloalkoxy, haloalkyl, halogen, hydroxy, hydroxyalkyl, mercapto, nitro, —NR_{A}R_{B}, or (NR_{A}R_{B})carbonyl;
R_{A} and R_{B} are independently hydrogen, alkyl, or alkylcarbonyl;
R₄ is 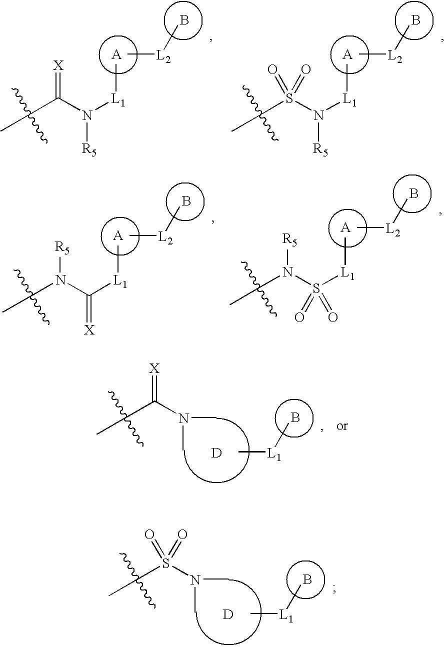
X is O or S;
R₅ is hydrogen or alkyl;
L₁ is a bond or alkylene;
L₂ is a bond or alkylene;
A is aryl, cycloalkyl, heteroaryl, or heterocycle;
B is aryl, cycloalkyl, heteroaryl, or heterocycle;
   D is heterocycle wherein the heterocycle is azetidinyl, azepanyl, aziridinyl, azocanyl, 1,1-dioxidothiomorpholinyl, morpholinyl, piperazinyl, piperidinyl, pyrrolidinyl, or thiomorpholinyl, wherein the heterocycle is optionally substituted with 1, 2, 3, or 4 substitutents independently selected from alkenyl, alkoxy, alkoxyalkyl, alkoxycarbonyl, alkyl, alkylcarbonyl, alkylcarbonyloxy, alkynyl, carboxy, cyano, formyl, haloalkoxy, haloalkyl, halo, hydroxy, hydroxyalkyl, mercapto, —NR_{A}R_{B}, or (NR_{A}R_{B})carbonyl.

In another embodiment of the present invention, compounds of formula (I) are disclosed wherein R₁ is aryl; R₄ is 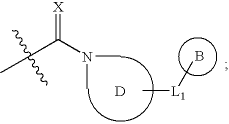
and R₂, R₃, X, D, B, and L₁ are as defined in formula (I).

In another embodiment of the present invention, compounds of formula (I) are disclosed wherein R₁ is aryl; R₄ is 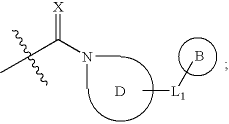
X is O; D is piperazinyl; L₁ is a bond; B is aryl; and R₂ and R₃ are as defined in formula (I).

In another embodiment of the present invention, compounds of formula (I) are disclosed wherein R₁ is aryl wherein the aryl is phenyl substituted with 1 halogen substituent wherein a preferred halogen substituent is —Cl; R₂ is hydrogen; R₃ is haloalkyl wherein a preferred haloalkyl is trifluoromethyl; R₄ is 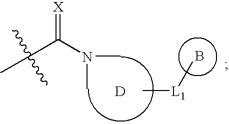
X is O; D is piperazinyl; L₁ is a bond; and B is aryl wherein the aryl is phenyl substituted with 1 halogen substituent wherein a preferred halogen substituent is —Cl.

In another embodiment of the present invention, compounds of formula (I) are disclosed wherein R₁ is aryl wherein the aryl is phenyl substituted with 1 halogen substituent wherein a preferred halogen substituent is —Cl; R₂ is hydrogen; R₃ is haloalkyl wherein a preferred haloalkyl is trifluoromethyl; R₄ is 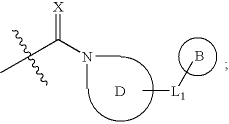
X is O; D is piperazinyl; L₁ is a bond; and B is cycloalkyl wherein the cycloalkyl is cyclohexyl.

In another embodiment of the present invention, compounds of formula (I) are disclosed wherein R₁ is aryl; R₄ is 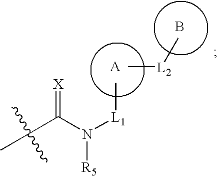
and R₂, R₃, R₅, X, A, B, L₁, and L₂ are as defined in formula (I).

In another embodiment of the present invention, compounds of formula (I) are disclosed wherein R₁ is aryl; R₄ is 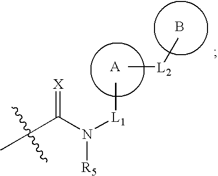
X is O; L₁ is a bond; A is piperidinyl; L₂ is alkylene; B is aryl; and R₅ is as defined in formula (I).

In another embodiment of the present invention, compounds of formula (I) are disclosed wherein R₁ is aryl wherein the aryl is phenyl substituted with 1 halogen substituent wherein a preferred substituent is —Cl; R₂ is hydrogen; R₃ is alkyl; R₄ is 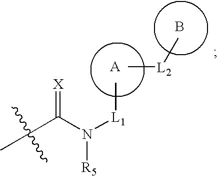
X is O; L₁ is a bond; A is piperidinyl; L₂ is alkylene; B is aryl wherein the aryl is phenyl; and R₅ is as defined in formula (I).

In another embodiment of the present invention, compounds of formula (I) are disclosed wherein R₁ is aryl wherein the aryl is phenyl substituted with 1 halogen substituent wherein a preferred substituent is —Cl; R₂ is hydrogen; R₃ is alkyl; R₄ is 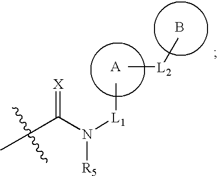
X is O; L₁ is a bond; A is piperidinyl; L₂ is alkylene wherein —CH₂— is preferred; B is aryl wherein the aryl is phenyl; and R₅ is hydrogen.

In another embodiment of the present invention, compounds of formula (II) are disclosed 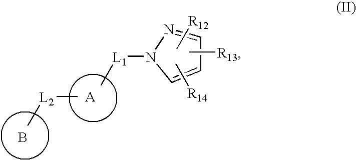
or a pharmaceutically acceptable salt, amide, ester, or prodrug thereof, wherein
R₁₂ and R₁₃ are independently hydrogen, alkenyl, alkoxy, alkoxyalkyl, alkoxycarbonyl, alkyl, alkylcarbonyl, alkylcarbonyloxy, alkylthio, alkynyl, aryl, arylalkyl, carboxy, cycloalkyl, cycloalkylalkyl, cyano, formyl, haloalkoxy, haloalkyl, halogen, hydroxy, hydroxyalkyl, mercapto, nitro, —NR_{A}R_{B}, or (NR_{A}R_{B})carbonyl;
R_{A} and R_{B} are independently hydrogen, alkyl, or alkylcarbonyl;
R₁₄ is 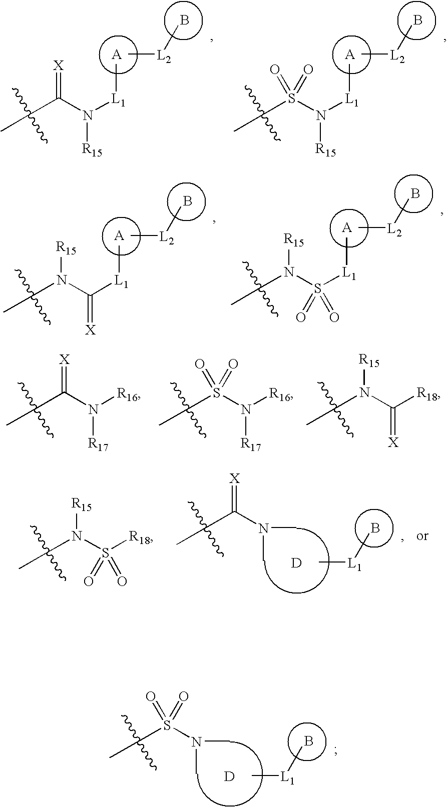
X is O or S;
R₁₅ is hydrogen or alkyl;
R₁₆ and R₁₇ are independently hydrogen, alkenyl, alkoxy, alkyl, aryl, arylalkyl, cycloalkyl, cycloalkylalkyl, heteroaryl, heteroarylalkyl, heterocycle, or heterocyclealkyl;
R₁₈ is alkyl, alkenyl, aryl, arylalkyl, cycloalkyl, cycloalkylalkyl, heteroaryl, heteroarylalkyl, heterocycle, heterocyclealkyl, —NR_{A}R_{B}, or (NR_{A}R_{B})alkyl;
L₁ is a bond or alkylene;
L₂ is absent, a bond, or alkylene;
A is aryl, cycloalkyl, heteroaryl, or heterocycle;
B is absent, aryl, cycloalkyl, heteroaryl, or heterocycle;
D is heterocycle wherein the heterocycle is azetidinyl, azepanyl, aziridinyl, azocanyl, 1,1-dioxidothiomorpholinyl, morpholinyl, piperazinyl, piperidinyl, pyrrolidinyl, or thiomorpholinyl, wherein the heterocycle is optionally substituted with 1, 2, 3, or 4 substitutents independently selected from alkenyl, alkoxy, alkoxyalkyl, alkoxycarbonyl, alkyl, alkylcarbonyl, alkylcarbonyloxy, alkynyl, carboxy, cyano, formyl, haloalkoxy, haloalkyl, halo, hydroxy, hydroxyalkyl, mercapto, —NR_{A}R_{B}, or (NR_{A}R_{B})carbonyl.

In another embodiment of the present invention, compounds of formula (II) are disclosed wherein L₁ is a bond; A is heterocycle; L₂ is alkylene; B is aryl; R₁₄ is 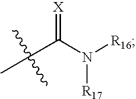
and R₁₂, R₁₃, R₁₆ and R₁₇ are as defined in formula (II).

In another embodiment of the present invention, compounds of formula (II) are disclosed wherein L₁ is a bond; A is heterocycle wherein the heterocycle is piperidinyl; L₂ is alkylene; B is aryl wherein the aryl is phenyl; R₁₄ is 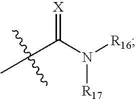
X is O; R₁₂ and R₁₆ are hydrogen; R₁₃ is haloalkyl; and R₁₇ is aryl wherein the aryl is phenyl substituted with 1 alkylsulfonyl substituent.

In another embodiment of the present invention, compounds of formula (II) are disclosed wherein L₁ is a bond; A is heterocycle; L₂ and B are absent; and R₁₄ is 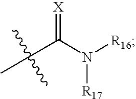
and R₁₂, R₁₃, R₁₆ and R₁₇ are as defined in formula (II).

In another embodiment of the present invention, compounds of formula (II) are disclosed wherein L₁ is a bond; A is heterocycle wherein the heterocycle is tetrahydropyran; L₂ and B are absent; R₁₄ is 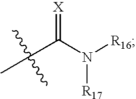
and R₁₂, R₁₃, R₁₆ and R₁₇ are as defined in formula (II).

In another embodiment of the present invention, compounds of formula (II) are disclosed wherein L₁ is a bond; A is heterocycle wherein the heterocycle is tetrahydropyran; L₂ and B are absent; R₁₄ is 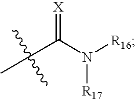
and R₁₂ and R₁₆ are hydrogen; R₁₃ is alkyl or haloalkyl; and R₁₇ is aryl.

Another embodiment of the present invention relates to pharmaceutical compositions comprising a therapeutically effective amount of a compound of formula (I) or a pharmaceutically acceptable salt, amide, ester, or prodrug thereof.

Another embodiment of the present invention relates to pharmaceutical compositions comprising a therapeutically effective amount of a compound of formula (II) or a pharmaceutically acceptable salt, amide, ester, or prodrug thereof.

Another embodiment of the present invention relates to a method for modulating PN3 in a host mammal comprising administering a therapeutically effective amount of a compound of formula (I) or a pharmaceutically acceptable salt, amide, ester, or prodrug thereof.

Another embodiment of the present invention relates to a method for modulating PN3 in a host mammal comprising administering a therapeutically effective amount of a compound of formula (II) or a pharmaceutically acceptable salt, amide, ester, or prodrug thereof.

Another embodiment of the present invention relates to a method for treating pain, in particular neuropathic pain, comprising administering a therapeutically effective amount of a compound of formula (I) or a pharmaceutically acceptable salt, amide, ester, or prodrug thereof.

Another embodiment of the present invention relates to a method for treating pain, in particular neuropathic pain, comprising administering a therapeutically effective amount of a compound of formula (II) or a pharmaceutically acceptable salt, amide, ester, or prodrug thereof.

Definition of Terms

As used throughout this specification and the appended claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl (vinyl), 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxysulfonyl” as used herein, means an alkoxy group, as defined herein, appended appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylene” means a divalent group derived from a straight or branched chain hydrocarbon of from 1 to 10 carbon atoms. Representative examples of alkylene include, but are not limited to, —CH₂—, —CH(CH₃)—, —C(CH₃)₂—, —CH₂CH₂—, —CH₂CH₂CH₂—, —CH₂CH₂CH₂CH₂—, and —CH₂CH(CH₃)CH₂—.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” as used herein, means a phenyl group, or a bicyclic or a tricyclic fused ring system wherein one or more of the fused rings is a phenyl group. Bicyclic fused ring systems are exemplified by a phenyl group fused to a cycloalkyl group, as defined herein, or another phenyl group. Tricyclic fused ring systems are exemplified by a bicyclic fused ring system fused to a cycloalkyl group, as defined herein, or another phenyl group. Representative examples of aryl include, but are not limited to, anthracenyl, azulenyl, fluorenyl, indanyl, indenyl, naphthyl, phenyl and tetrahydronaphthyl.

The aryl groups of this invention can be substituted with 1, 2, or 3 substituents independently selected from alkenyl, alkoxy, alkoxyalkyl, alkoxycarbonyl, alkoxysulfonyl, alkyl, alkylcarbonyl, alkylcarbonyloxy, alkylsulfonyl, alkynyl, carboxy, cyano, formyl, haloalkoxy, haloalkyl, halo, hydroxy, hydroxyalkyl, mercapto, —NR_{A}R_{B}, (NR_{A}R_{B})carbonyl, or (NR_{A}R_{B})sulfonyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “carbonyl” as used herein, means a —C(O)— group.

The term “carboxy” as used herein, means a —CO₂H group.

The term “cyano” as used herein, means a —CN group.

The term “cycloalkyl” as used herein, means a saturated cyclic hydrocarbon group containing from 3 to 8 carbons. Examples of cycloalkyl include cyclopropyl, cyclobutyl, cyclopentyl, cyclohexyl, cycloheptyl, and cyclooctyl.

The cycoalkyl groups of the present invention are optionally substituted with 1, 2, 3, or 4 substituents selected from

The term “cycloalkylalkyl” as used herein, means a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “formyl” as used herein, means a —C(O)H group.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl,” as used herein, refers to an aromatic five- or six-membered ring wherein 1, 2, 3, or 4 heteroatoms are independently selected from N, O, or S. The five membered rings have two double bonds and the six membered rings have three double bonds. The heteroaryl groups are connected to the parent molecular moiety through a carbon or nitrogen atom. The term “heteroaryl” also includes bicyclic systems where a heteroaryl ring is fused to a phenyl group, a monocyclic cycloalkyl group, as defined herein, a heterocycle group, as defined herein, or an additional heteroaryl group; and tricyclic systems where a bicyclic system is fused to a phenyl group, a monocyclic cycloalkyl group, as defined herein, a heterocycle group, as defined herein, or an additional heteroaryl group. Representative examples of heteroaryl include, but are not limited to, benzothienyl, benzoxadiazolyl, cinnolinyl, dibenzofuranyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, tetrazolyl, thiadiazolyl, thiazolyl, thienopyridinyl, thienyl, triazolyl, and triazinyl.

The heteroaryl groups of the present invention are substituted with 0, 1, 2, 3, or 4 substituents independently selected from alkenyl, alkoxy, alkoxyalkyl, alkoxycarbonyl, alkoxysulfonyl, alkyl, alkylcarbonyl, alkylcarbonyloxy, alkylsulfonyl, alkynyl, carboxy, cyano, formyl, haloalkoxy, haloalkyl, halo, hydroxy, hydroxyalkyl, mercapto, —NR_{A}R_{B}, (NR_{A}R_{B})carbonyl, or (NR_{A}R_{B})sulfonyl.

The term “heteroarylalkyl” as used herein, means a heteroaryl, as defined herein, appended to the parent molecular moiety through an alkyl group, as defined herein. Representative examples of heteroarylalkyl include, but are not limited to, pyridin-3-ylmethyl and 2-pyrimidin-2-ylpropyl.

The term “heterocycle,” as used herein, refers to a three, four, five, six, seven or eight membered ring containing one, two, or three heteroatoms independently selected from the group consisting of nitrogen, oxygen, and sulfur. The three membered ring has zero double bonds. The four and five membered ring has zero or one double bonds. The six membered ring has zero, one, or two double bonds. The seven and eight membered rings have zero, one, two, or three double bonds. The heterocycle groups of the present invention can be attached to the parent molecular moiety through a carbon atom or a nitrogen atom. Representative examples of heterocycle include, but are not limited to, azetidinyl, azepanyl, aziridinyl, azocanyl, morpholinyl, piperazinyl, piperidinyl, pyrrolidinyl, tetrahydropyranyl, and thiomorpholinyl.

The heterocycles of the present invention are substituted with 0, 1, 2, 3, or 4 substituents independently selected from alkenyl, alkoxy, alkoxyalkyl, alkoxycarbonyl, alkoxysulfonyl, alkyl, alkylcarbonyl, alkylcarbonyloxy, alkylsulfonyl, alkynyl, carboxy, cyano, formyl, haloalkoxy, haloalkyl, halo, hydroxy, hydroxyalkyl, mercapto, oxo, —NR_{A}R_{B}, (NR_{A}R_{B})carbonyl, or (NR_{A}R_{B})sulfonyl.

The term “heterocyclealkyl” as used herein, means a heterocycle, as defined herein, appended to the parent molecular moiety through an alkyl group, as defined herein. Representative examples of heterocyclealkyl include, but are not limited to, pyridin-3-ylmethyl and 2-pyrimidin-2-ylpropyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “nitro” as used herein, means a —NO₂ group.

The term “—NR_{A}R_{B}” as used herein, means two groups, R₁ and R₂, which are appended to the parent molecular moiety through a nitrogen atom. R₁ and R₂ are each independently hydrogen, alkyl, or alkylcarbonyl. Representative examples of —NR_{A}R_{B} include, but are not limited to, amino, methylamino, acetylamino, and acetylmethylamino.

The term “(NR_{A}R_{B})carbonyl” as used herein, means a —NR_{A}R_{B} group, as defined herein, appended to the parent molecular moiety through a carbonyl group, as defined herein. Representative examples of (NR_{A}R_{B})carbonyl include, but are not limited to, aminocarbonyl, (methylamino)carbonyl, (dimethylamino)carbonyl, and (ethylmethylamino)carbonyl.

The term “(NR_{A}R_{B})sulfonyl” as used herein, means a —NR_{A}R_{B} group, as defined herein, appended to the parent molecular moiety through a sulfonyl group, as defined herein. Representative examples of (NR_{A}R_{B})sulfonyl include, but are not limited to, aminosulfonyl, (methylamino)sulfonyl, (dimethylamino)sulfonyl, and (ethylmethylamino)sulfonyl.

The term “oxo” as used herein, means a ═O moiety.

The term “sulfonyl” as used herein, means a —SO₂— group.

Compounds of the present invention can exist as stereoisomers, wherein asymmetric or chiral centers are present. Stereoisomers are designated (R) or (S), depending on the configuration of substituents around the chiral carbon atom. The terms (R) and (S) used herein are configurations as defined in IUPAC 1974 Recommendations for Section E, Fundamental Stereochemistry, Pure Appl. Chem., (1976), 45: 13-30. The present invention contemplates various stereoisomers and mixtures thereof and are specifically included within the scope of this invention. Stereoisomers include enantiomers, diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution, a technique well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) direct separation of the mixture of optical enantiomers on chiral chromatographic columns, or (3) formation of a diastereomeric salt followed by selective recrystallization of one of the diastereomeric salts.

Abbreviations

Abbreviations which have been used in the descriptions of the Schemes and the Examples that follow are: Ac for acetyl, DMAP for N,N-dimethylaminopyridine, Et for ethyl

Preparation of Compounds of the Present Invention

The compounds and processes of the present invention will be better understood in connection with the following synthetic Schemes and Examples which illustrate a means by which the compounds of the present invention can be prepared. Further, all citations herein are incorporated by reference.

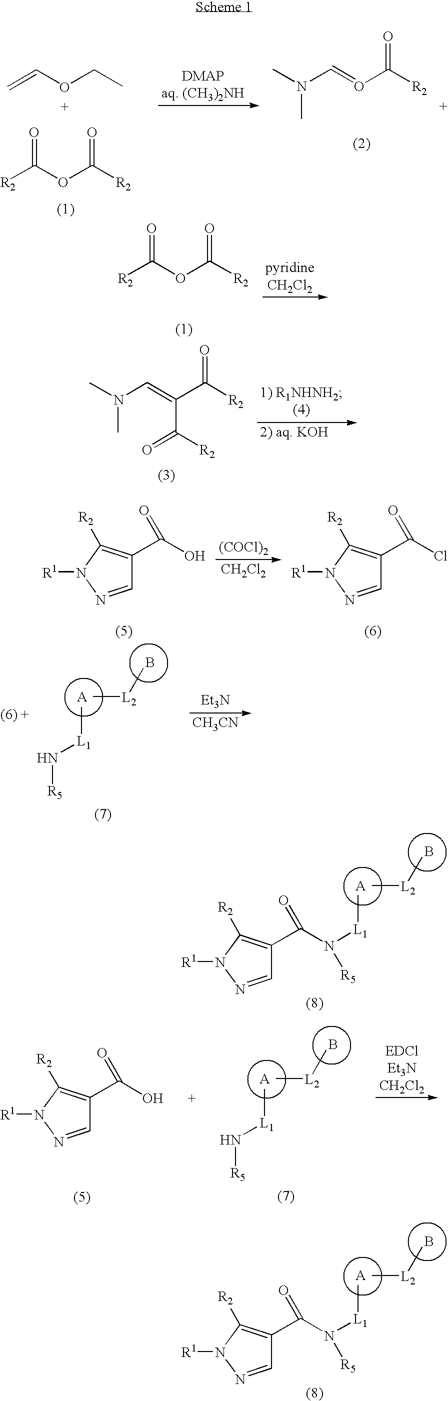

Pyrazoles of general formula (8), wherein R₁, R₅, L₁, A, L₂, and B are as defined in formula (I) and R₂ is alkenyl, alkyl, alkynyl, aryl, arylalkyl, cycloalkyl, cycloalkylalkyl, haloalkoxy, or haloalkyl, can be prepared as described in Scheme 1. Ethyl vinyl ether can be treated with an anhydride of general formula (1), aqueous methylamine, and N,N-dimethylaminopyridine to provide enaminones of general formula (2) as described in Mellor, et. al., Tetrahedron, 56:7255-7267 (2000). Enaminones of general formula (2) can be treated with an anhydride of general formula (1) and pyridine to provide compounds of general formula (3). Compounds of general formula (3) can be treated with hydrazines of general formula (4) to provide pyrazoles which can be treated with aqueous base including, but not limited to, potassium hydroxide or sodium hydroxide to provide acids of general formula (5). Acids of general formula (5) can be treated with oxalyl chloride to provide acid chlorides of general formula (6). Acid chlorides of general formula (6) can be treated with amines of general formula (7) and triethylamine or diisopropylethylamine to provide pyrazoles of general formula (8).

Acids of general formula (5) can also be treated with an amine of general formula (7), a carbodiimide including, but not limited to, 1-(3-dimethylaminopropyl)-3-ethylcarbodiimide hydrochloride (EDCI) or dicyclohexycarbodiimide (DCC), and triethylamine to provide pyrazoles of general formula (8).

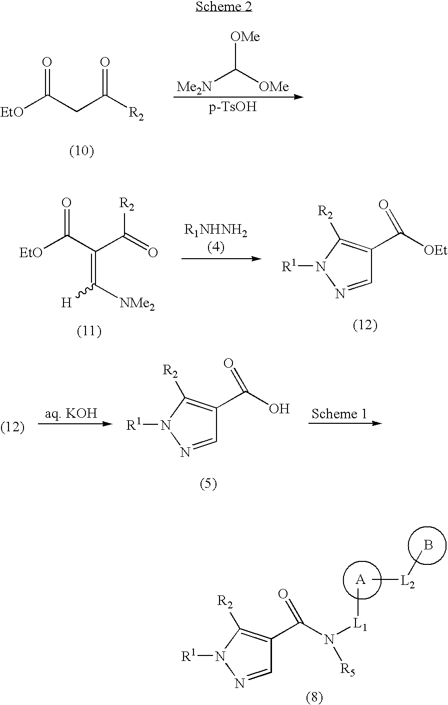

Pyrazoles of general formula (8), wherein R₁, R₅, L₁, A, L₂, and B are as defined in formula (I) and R₂ is alkenyl, alkyl, alkynyl, aryl, arylalkyl, cycloalkyl, cycloalkylalkyl, haloalkoxy, or haloalkyl, can be prepared as described in Scheme 2. Keto esters of general formula (10) can be treated with N-(dimethoxymethyl)-N,N-dimethylamine and an acid including, but not limited to, para-toluenesulfonic acid to provide compounds of general formula (11). Compounds of general formula (11) can be treated with hydrazines of general formula (4) to provide esters of general formula (12) as described in Menozzi, et. al., J. Het. Chem., 24:1669 (1987). Esters of general formula (12) can be treated with aqueous base including, but not limited to, potassium hydroxide or sodium hydroxide to provide acids of general formula (5). Acids of general formula (5) can be processed as described in Scheme 1 to provide pyrazoles of general formula (8).

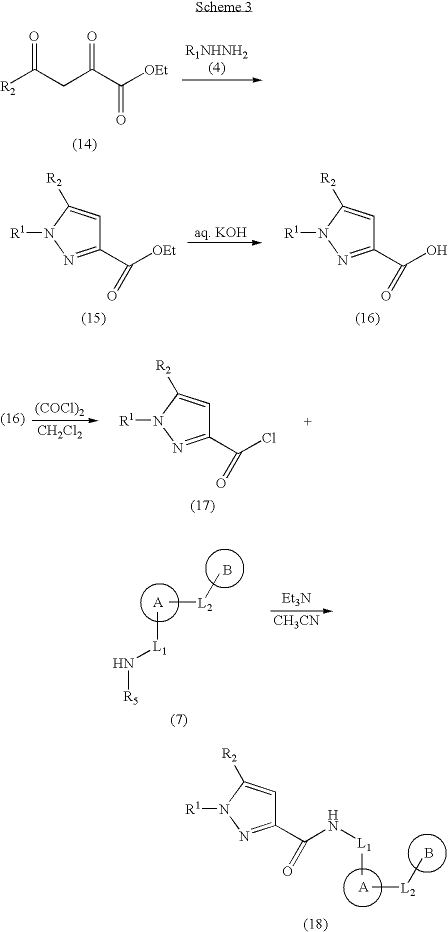

Pyrazoles of general formula (18), wherein R₁, R₅, L₁, A, L₂, and B are as defined in formula (I) and R₂ is alkenyl, alkyl, alkynyl, aryl, arylalkyl, cycloalkyl, cycloalkylalkyl, haloalkoxy, or haloalkyl, can be prepared as described in Scheme 3. Keto esters of general formula (14) can be treated with hydrazines of general formula (4) to provide esters of general formula (15) as described in Kordik, et. al., Bio. Med. Chem. Let., 11:2287-2290 (2001). Esters of general formula (15) can be treated with aqueous base including, but not limited to, potassium hydroxide or sodium hydroxide to provide acids of general formula (16). Acids of general formula (16) can be treated with oxalyl chloride to provide acid chlorides of general formula (17). Acid chlorides of general formula (17) can be treated with amines of general formula (7) and triethylamine or diisopropylethylamine to provide pyrazoles of general formula (18).

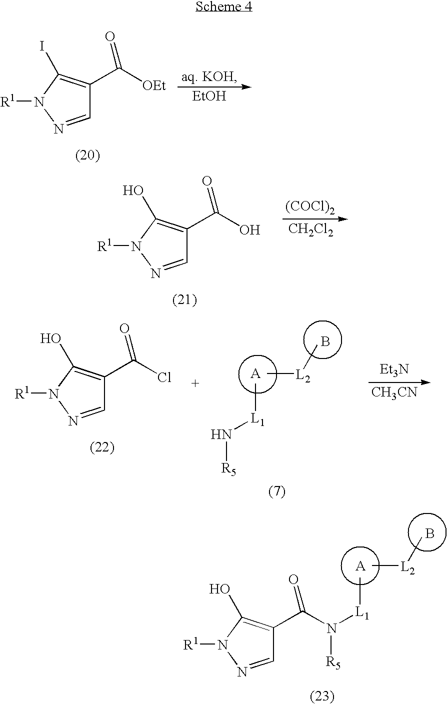

Pyrazoles of general formula (23), wherein R₁, R₅, L₁, A, L₂, and B are as defined in formula (I), can be prepared as described in Scheme 4. Esters of general formula (20), prepared using the procedure described in Beck, et. al., J. Het. Chem., 267:267-270 (1987), can be treated with aqueous base including, but not limited to, potassium hydroxide or sodium hydroxide to provide acids of general formula (21). Acids of general formula (21) can be treated with oxalyl chloride to provide acid chlorides of general formula (22). Acid chlorides of general formula (22) can be treated with amines of general formula (7) and triethylamine or diisopropylethylamine to provide pyrazoles of general formula (23).

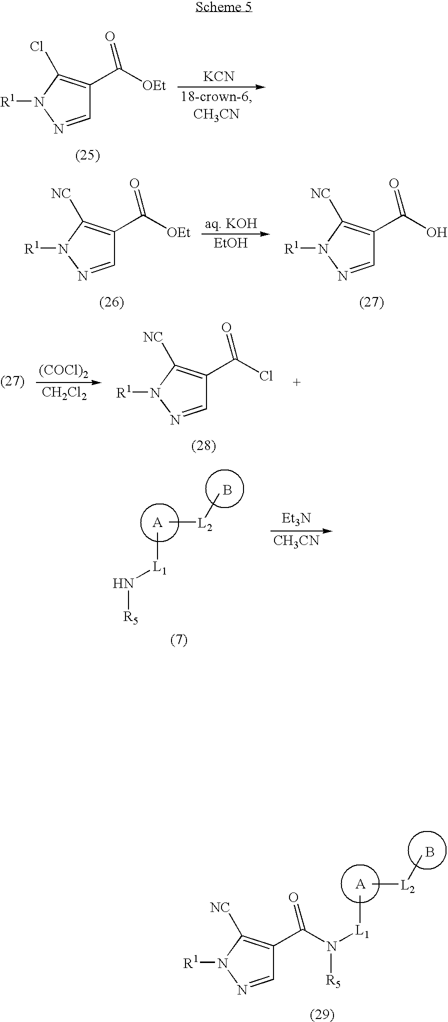

Pyrazoles of general formula (29), wherein R₁, R₅, L₁, A, L₂, and B are as defined in formula (I), can be prepared as described in Scheme 5. Esters of general formula (25) can be treated with potassium cyanide or sodium cyanide to provide esters of general formula (26). Esters of general formula (26) can be treated with aqueous base including, but not limited to, potassium hydroxide or sodium hydroxide to provide acids of general formula (27). Acids of general formula (27) can be treated with oxalyl chloride to provide acid chlorides of general formula (28). Acid chlorides of general formula (28) can be treated with amines of general formula (7) and triethylamine or diisopropylethylamine to provide pyrazoles of general formula (29).

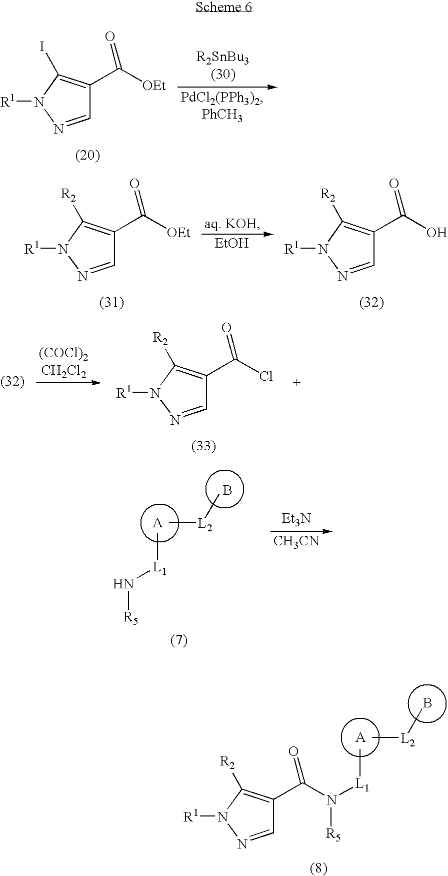

Pyrazoles of general formula (8), wherein R₁, R₅, L₁, A, L₂, and B are as defined in formula (I) and R₂ is alkenyl, alkyl, alkylcarbonyl, alkynyl, or aryl, can be prepared as described in Scheme 6. Esters of general formula (20) can be treated with a palladium catalyst including, but not limited to, dichlorobis[tri(o-tolyl)phosphine]palladium(II) and a tin compound of general formula (30) wherein R₂ is alkenyl, alkyl, alkynyl, aryl, or a vinyl ether including, but not limited to, ethoxyvinyl to provide esters of general formula (31). Esters of general formula (31) can be treated with aqueous base including, but not limited to, potassium hydroxide or sodium hydroxide to provide acids of general formula (32). Acids of general formula (32) can be treated with oxalyl chloride to provide acid chlorides of general formula (33). Acid chlorides of general formula (33) can be treated with amines of general formula (7) and triethylamine or diisopropylethylamine to provide pyrazoles of general formula (8) wherein R₂ is alkenyl, alkyl, alkylcarbonyl, alkynyl, or aryl.

It is to be understood that the order of the reactions in the synthesis exemplified in Scheme 6 can be rearranged. For example, the tin coupling reaction can be executed as the last step to provide pyrazoles of general formula (8) wherein R₂ is alkenyl, alkyl, alkylcarbonyl, alkynyl, or aryl.

### EXAMPLE 1

### 1-(3-chlorophenyl)-4-{[1-(4-chlorophenyl)-5-(trifluoromethyl)-1H-pyrazol-4-yl]carbonyl}piperazine hydrochloride

### EXAMPLE 1A

### 4-Dimethylamino-1,1,1-trifluoro-but-3-en-2-one

Trifluoroacetic anhydride (2.0 g, 9.5 mmol.) was dissolved in dichloromethane (20 mL) and the mixture was cooled to 0° C. with an ice bath. Ethyl vinyl ether (0.69 g, 9.5 mmol) and a catalytic amount of DMAP were added and the mixture was stirred for 30 min. The ice bath was removed and the mixture was warmed to ambient temperature and stirred for an additional 2 h. The mixture was cooled to −5° C. with an ice/brine bath and 40% (w/v) aqueous dimethylamine (3.5 mL) was added. The mixture was stirred at −5° C. for 10 min then diluted with dichloromethane (20 mL). The organic phase was washed with brine (20 mL), dried over Na₂SO₄, and filtered through a ½″ plug of silica gel. The silica gel plug was washed with EtOAc (150 mL) and the mixture was concentrated under reduced pressure and recrystallized from cold Et₂O/hexanes (1:50) to provide 1.0 g of the desired product. MS (DCI/NH₃) M/Z 168 (M+H)⁺.

### EXAMPLE 1B

### 3-Dimethylaminomethylene-1,1,1,5,5,5-hexafluoro-pentane-2,4-dione

The product from Example 1A (1.0 g, 6.0 mmol) was dissolved in dichloromethane (7 mL) and cooled to −5° C. with an ice/brine bath. Pyridine (0.61 g, 7.7 mmol) and trifluoroacetic anhydride (1.6 g, 7.7 mmol) were added and the mixture was stirred at −5° C. for 10 min. The mixture was warmed to ambient temperature, diluted with water (75 mL) and extracted twice with dichloromethane (75 mL). The combined extracts were concentrated under reduced pressure to provide 1.3 g of the desired product. MS (DCI/NH₃) m/z 264 (M+H)⁺.

### EXAMPLE 1C

### 1-(4-Chlorophenyl)-5-trifluoromethyl-1H-pyrazole-4-carboxylic acid

4-Chlorophenylhydrazine hydrochloride, the product from Example 1B, and triethylamine were dissolved in acetonitrile (12 mL) and stirred at ambient temperature for 16 h. The solvent was removed under reduced pressure and the crude material was dissolved in EtOAc/hexanes (1:1) and filtered through a ½″ silica gel frit. The solvent was removed under reduced pressure and the crude material was dissolved in 1,4-dioxane (15 mL). Aqueous KOH was added and the mixture was heated at reflux for 30 min. The mixture was cooled to ambient temperature diluted with 2N HCl (6 mL), and extracted twice with EtOAc (20 mL). The combined extracts were washed with brine, dried over Na₂SO₄, and concentrated under reduced pressure. The crude material was triturated with hexanes to provide the desired product.

MS (DCI/NH₃) m/z 291 (M+H)⁺.

### EXAMPLE 1D

### 1-(3-chlorophenyl)-4-{[1-(4-chlorophenyl)-5-(trifluoromethyl)-1H-pyrazol-4-yl]carbonyl}piperazine hydrochloride

A solution of the product from Example 1C in dichloromethane (15 mL) was treated with oxalyl chloride and a catalytic amount of DMF. The mixture was allowed to stir at ambient temperature for 1 h and the solvent and excess oxalyl chloride were removed under reduced pressure. The material was re-dissolved in dichloromethane (15 mL) and treated with 1-(3-chlorophenyl)piperazine and triethylamine and allowed to stir at ambient temperature for 1 h. The mixture was diluted with NaHCO₃ (10 mL) and extracted twice with EtOAc (15 mL). The combined extracts were dried and concentrated under reduced pressure and the material was purified by silica gel chromatography. The purified material was dissolved in Et₂O and treated with ethanolic HCl to obtain the salt. MS (DCI/NH₃) m/z 469 (M−HCl)⁺. ¹H NMR (DMSO-d₆) δ 8.09 (d, 1H, J=0.7 Hz), 7.69 (d, 2H, J=9.2 Hz), 7.63 (d, 2H, J=8.8 Hz), 7.24 (t, 1H, J=8.1 Hz), 7.00 (t, 1H, J=2.2 Hz), 6.93 (dd, 1H, J=8.5, 0.7 Hz), 6.83 (dd, 1H, J=7.8, 0.7 Hz), 3.77 (br s, 2H), 3.53 (br s, 2H), 3.26 (br s, 2H), 3.19 (br s, 2H).

### EXAMPLE 2

### 1-{[1-(4-chlorophenyl)-5-(trifluoromethyl)-1H-pyrazol-4-yl]carbonyl}-4-cyclohexylpiperazine

A solution of the product from Example 1C and N-cyclohexylpiperazine were processed as described in Example 1D to provide the desired product. MS (DCI/NH₃) m/z 441 (M−HCl)⁺. ¹H NMR (DMSO-d₆) δ 8.03 (d, 1H, J=0.7 Hz), 7.68 (d, 2H, J=8.8 Hz), 7.61 (d, 2H, J=8.8 Hz), 3.60 (br s, 2H), 3.34 (br s, 2H), 2.51 (br s, 2H), 2.45 (br s, 2H), 2.27 (br s, 2H), 1.79-1.68 (m, 4H), 1.61-1.52 (m, 1H), 1.26-1.00 (m, 5H).

### EXAMPLE 3

### 1-(3-chlorophenyl)-N-[3-(methylsulfonyl)phenyl]-5-(trifluoromethyl)-1H-pyrazole-4-carboxamide

### EXAMPLE 3A

### 1-(3-Chlorophenyl)-5-trifluoromethyl-1H-pyrazole-4-carboxylic acid

3-Chlorophenylhydrazine hydrochloride, the product from Example 1B, and triethylamine were processed as described in Example 1C to provide the desired product.

MS (ESI−) m/z 289 (M−H)⁻.

### EXAMPLE 3B 1-(3-chlorophenyl)-N-[3-(methylsulfonyl)phenyl]-5-(trifluoromethyl)-1H-pyrazole-4-carboxamide

A solution of the product from Example 3A and 3-(methanesulfonyl)aniline were processed as described in Example 1D to provide the desired product. MS (ESI+) m/z 444 (M+H)⁺; ¹H NMR (DMSO-d₆) δ 10.89 (s, 1H), 8.40 (d, 1H, J=0.7 Hz), 8.37-8.34 (m, 1H), 8.02 (dt, 1H, J=7.1, 2.2 Hz), 7.76-7.62 (m, 5H), 7.56 (dt, 1H, J=7.8, 1.5 Hz), 3.23 (s, 3H).

### EXAMPLE 4

### 1-(3-methylphenyl)-N-[3-(methylsulfonyl)phenyl]-5-(trifluoromethyl)-1H-pyrazole-4-carboxamide

### EXAMPLE 4A

### 1-(3-Methylphenyl)-5-trifluoromethyl-1H-pyrazole-4-carboxylic acid

3-Methylphenylhydrazine hydrochloride, the product from Example 1B, and triethylamine were processed as described in Example 1C to provide the desired product. MS (ESI−) m/z 269 (M−H)⁻.

### EXAMPLE 4B

### 1-(3-methylphenyl)-N-[3-(methylsulfonyl)phenyl]-5-(trifluoromethyl)-1H-pyrazole-4-carboxamide

A solution of the product from Example 4A and 3-(methanesulfonyl)aniline were processed as described in Example 1D to provide the desired product. MS (ESI+) m/z 441 (M+NH₄)⁺. ¹H NMR (DMSO-d₆) δ 10.88 (s, 1H), 8.38-8.34 (m, 2H), 8.01 (dt, 1H, J=6.8, 2.4 Hz), 7.72-7.63 (m, 2H), 7.53-7.41 (m, 2H), 7.38-7.30 (m, 2H), 3.23 (s, 3H), 2.42 (s, 3H).

### EXAMPLE 5

### 1-(4-methylphenyl)-N-[3-(methylsulfonyl)phenyl]-5-(trifluoromethyl)-1H-pyrazole-4-carboxamide

### EXAMPLE 5A

### 1-(4-Methylphenyl)-5-trifluoromethyl-1H-pyrazole-4-carboxylic acid

4-Methylphenylhydrazine hydrochloride, the product from Example 1B, and triethylamine were processed as described in Example 1C to provide the desired product.

MS (ESI−) m/z 269 (M−H)⁻.

### EXAMPLE 5B

### 1-(4-methylphenyl)-N-[3-(methylsulfonyl)phenyl]-5-(trifluoromethyl)-1H-pyrazole-4-carboxamide

A solution of the product from Example 5A and 3-(methanesulfonyl)aniline were processed as described in Example 1D to provide the desired product. MS (ESI+) m/z 441 (M+NH₄)⁺. ¹H NMR (DMSO-d₆) δ 10.88 (s, 1H), 8.37-8.34 (m, 1H), 8.33 (d, 1H, J=0.7 Hz), 8.00 (dt, 1H, J=7.1, 2.2 Hz), 7.71-7.64 (m, 2H), 7.43-7.40 (m, 4H), 3.23 (s, 3H), 2.42 (s, 3H).

### EXAMPLE 6

### 1-(2-methoxyphenyl)-5-(trifluoromethyl)-N-[3-(trifluoromethyl)benzyl]-1H-pyrazole-4-carboxamide

### EXAMPLE 6A

### 1-(2-Methoxyphenyl)-5-trifluoromethyl-1H-pyrazole-4-carboxylic acid

2-Methoxyphenylhydrazine hydrochloride, the product from Example 1B, and triethylamine were processed as described in Example 1C to provide the desired product.

MS (ESI−) m/z 285 (M−H)⁻.

### EXAMPLE 6B

### 1-(2-methoxyphenyl)-5-(trifluoromethyl)-N-[3-(trifluoromethyl)benzyl]-1H-pyrazole-4-carboxamide

A solution of the product from Example 6A and 3-trifluoromethylbenzyl amine were processed as described in Example 1D to provide the desired product. MS (ESI+) m/z 444 (M+H)⁺. ¹H NMR (DMSO-d₆) δ 9.16 (t, 1H, J=5.9 Hz), 8.19 (d, 1H, J=0.7 Hz), 7.69-7.52 (m, 5H), 7.42 (dd, 1H, J=7.8, 1.7 Hz), 7.26 (dd, 1H, J=8.5, 1.0 Hz), 7.10 (td, 1H, J=7.8, 1.4 Hz), 4.54 (d, 2H, J=5.8 Hz), 3.76 (s, 3H).

### EXAMPLE 7

### N-[2-(4-chlorophenyl)ethyl]-1-(2-methoxyphenyl)-5-(trifluoromethyl)-1H-pyrazole-4-carboxamide

A solution of the product from Example 6A and 2-(4-chlorophenyl)ethylamine were processed as described in Example 1D to provide the desired product. MS (ESI+) m/z 424 (M+H)⁺; ¹H NMR (DMSO-d₆) δ 8.58 (t, 1H, J=5.8 Hz), 8.04 (d, 1H, J=0.7 Hz), 7.56 (ddd, 1H, J=9.2, 7.5, 1.7 Hz), 7.40 (dd, 1H, J=7.8, 1.7 Hz), 7.36 (d, 2H, J=8.5 Hz), 7.31-7.23 (m, 3H), 7.10 (td, 1H, J=7.5, 1.0 Hz), 3.76 (s, 3H), 3.45 (q, 2H, J=6.7 Hz), 2.82 (t, 2H, J=7.1 Hz).

### EXAMPLE 8

### 1-cyclohexyl-5-(trifluoromethyl)-N-[3-(trifluoromethyl)benzyl]-1H-pyrazole-4-carboxamide

### EXAMPLE 8A

### 1-Cyclohexyl-5-trifluoromethyl-1H-pyrazole-4-carboxylic acid

Cyclohexylhydrazine hydrochloride, the product from Example 1B, and triethylamine were processed as described in Example 1C to provide the desired product. MS (ESI+) m/z 281 (M+NH₄)⁺.

### EXAMPLE 8B

### 1-cyclohexyl-5-(trifluoromethyl)-N-[3-(trifluoromethyl)benzyl]-1H-pyrazole-4-carboxamide

A solution of the product from Example 8A and 3-trifluoromethylbenzyl amine were processed as described in Example 1D to provide the desired product.

MS (ESI+) m/z 420 (M+H)⁺; ¹H NMR (DMSO-d₆) δ 9.06 (t, 1H, J=5.9 Hz), 7.91 (s, 1H), 7.66-7.53 (m, 4H), 4.49 (d, 2H, J=6.1 Hz), 4.33-4.18 (m, 1H), 1.97-1.77 (m, 6H), 1.73-1.60 (m, 1H), 1.51-1.31 (m, 2H), 1.29-1.13 (m, 1H).

### EXAMPLE 9

### 1-cyclohexyl-N-[3-(methylsulfonyl)phenyl]-5-(trifluoromethyl)-1H-pyrazole-4-carboxamide

A solution of the product from Example 8A and 3-(methanesulfonyl)aniline were processed as described in Example 1D to provide the desired product. MS (DCI/NH₃) m/z 433 (M+NH₄)⁺; ¹H NMR (DMSO-d₆) δ 10.80 (s, 1H), 8.33 (s, 1H), 8.08 (s, 1H), 7.94 (dt, 1H, J=7.1, 2.0 Hz), 7.69-7.62 (m, 2H), 4.37-4.24 (m, 1H), 3.21 (s, 3H), 1.99-1.80 (m, 6H), 1.75-1.63 (m, 1H), 1.53-1.34 (m, 2H), 1.32-1.17 (m, 1H).

### EXAMPLE 10

### N-[2-(4-chlorophenyl)ethyl]-1-(7-chloroquinolin-4-yl)-5-(trifluoromethyl)-1H-pyrazole-4-carboxamide

### EXAMPLE 10A

### 1-(7-Chloro-quinolin-4-yl)-5-trifluoromethyl-1H-pyrazole-4-carboxylic acid

(7-Chloro-quinolin-4-yl)-hydrazine hydrochloride, the product from Example 1B, and triethylamine were processed as described in Example 1C to provide the desired product. MS (ESI+) m/z 342 (M+H)⁺.

### EXAMPLE 10B

### N-[2-(4-chlorophenyl)ethyl]-1-(7-chloroquinolin-4-yl)-5-(trifluoromethyl)-1H-pyrazole-4-carboxamide

A solution of the product from Example 10A and 2-(4-chlorophenyl)ethylamine were processed as described in Example 1D to provide the desired product.

MS (ESI+) m/z 479 (M)⁺; ¹H NMR (DMSO-d₆) δ 9.18 (d, 1H, J=4.7 Hz), 8.74 (t, 1H, J=5.8 Hz), 8.32 (d, 1H, J=2.0 Hz), 8.30 (s, 1H), 7.86 (d, 1H, J=4.4 Hz), 7.78 (dd, 1H, J=8.8, 2.0 Hz), 7.41-7.28 (m, 5H), 3.50 (q, 2H, J=6.8 Hz), 2.85 (t, 2H, J=7.3 Hz).

### EXAMPLE 11

### 1-(1-benzylpiperidin-4-yl)-N-[3-(methylsulfonyl)phenyl]-5-(trifluoromethyl)-1H-pyrazole-4-carboxamide

### EXAMPLE 11A

### 1-(1-Benzylpiperidin-4-yl)-5-trifluoromethyl-1H-pyrazole-4-carboxylic acid

(1-Benzylpiperidin-4-yl)-hydrazine dihydrochloride, the product from Example 1B, and triethylamine were processed as described in Example 1C to provide the desired product.

MS (ESI+) m/z 354 (M+H)⁺.

### EXAMPLE 11B

### 1-(1-benzylpiperidin-4-yl)-N-[3-(methylsulfonyl)phenyl]-5-(trifluoromethyl)-1H-pyrazole-4-carboxamide

A solution of the product from Example 11A and 3-(methylsulfonyl)aniline were processed as described in Example 1D to provide the desired product. MS (ESI+) m/z 506 (M)⁺; ¹H NMR (DMSO-d₆) δ 10.84 (br s, 1H), 10.38 (br s, 1H), 8.32 (br s, 1H), 8.16 (br s, 1H), 7.94 (br d, 1H, J=7.6 Hz), 7.67-7.55 (m, 4H), 7.52-7.46 (m, 2H), 4.76 (m, 1H), 4.32 (m, 2H), 3.52-3.33 (m, 4H), 3.21 (s, 3H), 2.20-2.12 (m, 4H).

### EXAMPLE 12

### 1-(4-chlorophenyl)-5-methyl-N-[3-(trifluoromethyl)benzyl]-1H-pyrazole-4-carboxamide

### EXAMPLE 12A

### Ethyl 2-acetyl-3-(dimethylamino)acrylate

A mixture of ethyl acetoacetate (3.9 g, 30 mmol), p-toluenesulfonic acid monohydrate (catalytic amount) and N,N-dimethylformamide dimethyl acetal (5.3 g, 45 mmol) was stirred at 100° C. for 1.5 h. The mixture was distilled under vacuum (10 Torr). The distillate collected between 145° C. and 165° C. afforded 3.1 g of the desired compound as a yellowish oil. MS (ESI) m/z 186 (M+H)⁺; ¹H NMR (CDCl₃) δ 7.67 (s, 1H), 4.23 (q, 2H, J=7.1 Hz), 3.04 (s, 6H), 2.33 (s, 3H), 1.32 (t, 3H, J=7.1 Hz).

### EXAMPLE 12B

### 1-(4-Chlorophenyl)-5-methyl-1H-pyrazole-4-carboxylic acid ethyl ester

An acetonitrile solution of the product from Example 12A (0.87 g, 4.7 mmol) and triethylamine (650 μL, 4.70 mmol) was added to a suspension of 4-chlorophenylhydrazine hydrochloride (0.84 g, 4.7 mmol) in acetonitrile (20 mL). The reaction mixture was stirred at 25° C. for 10 h. The solution was concentrated and the residue was purified by silica gel flash column chromatography (elution with 14% ethyl acetate/hexanes) to afford 1.22 g of the desired product as a brown oil. MS (ESI) m/z 265 (M+H)⁺; ¹H NMR (CDCl₃) δ 8.02 (s, 1H), 7.51-7.46 (m, 2H), 7.40 7.35 (m, 2H), 4.33 (q, 2H, J=7.1 Hz), 2.57 (s, 3H), 1.38 (t, 3H, J=7.1 Hz).

### EXAMPLE 12C

### 1-(4-Chlorophenyl)-5-methyl-1H-pyrazole-4-carboxylic acid

The product from Example 12B (1.2 g, 4.5 mmol) was dissolved in methanol (10 mL) and treated with a solvent mixture of THF (12 mL), 20% KOH (12 mL) and methanol (12 mL). The solution was stirred at 25° C. for 10 h. The reaction mixture was then diluted with ethyl acetate (100 mL) and was partitioned between ethyl acetate (150 mL) and water (300 mL). The aqueous layer was acidified to pH 2 and repartitioned between dichloromethane (200 mL) and water (250 mL). The organic layer was dried (sodium sulfate) and concentrated in vacuo to afford 1.1 g of the desired product as a white solid. MS (DCI/NH₃) m/z 237 (M+H)⁺; ¹H NMR (CDCl₃) δ 8.10 (s, 1H), 7.52-7.47 (m, 2H), 7.42-7.37 (m, 2H), 4.78 (s, 1H), 2.59 (s, 3H).

### EXAMPLE 12D

### 1-(4-Chlorophenyl)-5-methyl-1H-pyrazole-4-carbonyl chloride

The product from Example 12C (570 mg, 2.40 mmol) in dichloromethane (30 mL) was treated with oxalyl chloride (230 μL, 2.60 mmol), catalyzed by one drop of DMF. The reaction mixture was stirred at 25° C. for 3 h and the solvent was evaporated to afford 610 mg of the title compound as a white solid.

### EXAMPLE 12E

### 1-(4-chlorophenyl)-5-methyl-N-[3-(trifluoromethyl)benzyl]-1H-pyrazole-4-carboxamide

A solution of the product from Example 12D and 4-cyclohexylpiperidine were processed as described in Example 1D to provide the desired product. MS (ESI) m/z 394 (M+H)⁺; ¹H NMR (CDCl₃) δ 8.77 (t, 1H, J=6.1 Hz), 8.17 (s, 1H), 7.69-7.54 (m, 8H), 4.53 (d, 2H, J=5.8 Hz), 2.52 (s, 3H).

### EXAMPLE 13

### N-(1-benzylpiperidin-4-yl)-1-(4-chlorophenyl)-5-methyl-1H-pyrazole-4-carboxamide

A solution of the product from Example 12D and 1-benzylpiperidin-4-ylamine were processed as described in Example 1D to provide the desired product. MS (ESI) m/z 409 (M+H)⁺; ¹H NMR (DMSO-d₆) δ 8.13 (s, 1H), 7.85 (d, 1H, J=7.8 Hz), 7.63-7.52 (m, 4H), 7.36-7.21 (m, 5H), 3.81-3.68 (m, 1H), 3.47 (s, 2H), 2.50 (s, 3H), 2.81 (d, 2H, J=11.5 Hz), 2.01 (t, 2H, J=11.5 Hz), 1.77 (d, 2H, J=12.1 Hz), 1.54 (q, 2H, J=11.7 Hz).

### EXAMPLE 14

### 1-(4-chlorophenyl)-N-[2-(4-chlorophenyl)ethyl]-5-methyl-1H-pyrazole-3-carboxamide

### EXAMPLE 14A

### 1-(4-Chlorophenyl)-5-methyl-1H-pyrazole-3-carboxylic acid ethyl ester

To an acetonitrile solution (25 mL) of 4-chlorophenylhydrazine hydrochloride (1.0 g, 5.6 mmol) and triethylamine (780 μL, 5.60 mmol) was added ethyl acetopyruvate (820 μL, 5.60 mmol). The solution was stirred at 25° C. for 12 h. The reaction mixture was diluted with dichloromethane (200 mL) and washed with water (250 mL). The organic layer was dried (sodium sulfate) and concentrated, and the residue was purified by silica gel flash column chromatography (elution with 25% ethyl acetate/hexanes) to afford 0.67 g of the title compound as a light yellow oil. MS (ESI) m/z 265 (M+H)⁺; ¹H NMR (DMSO-d₆) δ 7.62 (s, 4H), 6.77 (s, 1H), 4.29 (q, 2H, J=7.1 Hz), 2.34 (s, 3H), 1.30 (t, 3H, J=7.1 Hz).

### EXAMPLE 14B

### 1-(4-Chlorophenyl)-5-methyl-1H-pyrazole-3-carboxylic acid

A solution of the product from Example 14A were processed as described in Example 12C to provide the desired product. MS (ESI) m/z 237 (M+H)⁺.

### EXAMPLE 14C

### 1-(4-Chlorophenyl)-5-methyl-1H-pyrazole-3-carbonyl chloride

A solution of the product from Example 14B were processed as described in Example 12D to provide the desired product.

### EXAMPLE 14D

### 1-(4-chlorophenyl)-N-[2-(4-chlorophenyl)ethyl]-5-methyl-1H-pyrazole-3-carboxamide

A solution of the product from Example 14D and 4-chlorophenethylamine were processed as described in Example 1D to provide the desired product. MS (ESI) m/z 390 (M+H)⁺; ¹H NMR (DMSO-d₆) δ 10.23 (s, 1H), 8.40-8.36 (m, 2H), 8.13-8.06 (m, 1H), 7.68-7.58 (m, 6H), 3.22 (s, 3H), 2.58 (s, 3H).

### EXAMPLE 15

### 1-(4-chlorophenyl)-5-methyl-N-[3-(trifluoromethyl)benzyl]-1H-pyrazole-3-carboxamide

A solution of the product from Example 14D and 3-(trifluoromethyl)benzylamine were processed as described in Example 1D to provide the desired product. MS (ESI) m/z 394 (M+H)⁺; ¹H NMR (DMSO-d₆) δ 8.93 (t, 1H, J=6.3 Hz), 7.68-7.52 (m, 8H), 6.68 (d, 1H, J=1.0 Hz), 4.50 (d, 2H, J=6.4 Hz), 2.34 (d, 3H, J=0.7 Hz).

### EXAMPLE 16

### 1-(4-chlorophenyl)-5-methyl-N-[3-(methylsulfonyl)phenyl]-1H-pyrazole-3-carboxamide

A solution of the product from Example 14D and 3-(methanesulfonyl)aniline were processed as described in Example 1D to provide the desired product. MS (ESI) m/z 407 (M+NH₄)⁺; ¹H NMR (300 MHz, DMSO-d₆) δ 10.50 (s, 1H), 8.49 (q, 1H, J=1.2 Hz), 8.18-8.10 (m, 1H), 7.76-7.58 (m, 6H), 6.84 (d, 1H, J=1.0 Hz), 3.20 (d, 3H, J=1.0 Hz), 2.39 (d, 3H, J=0.7 Hz).

### EXAMPLE 17

### N-benzyl-1-(4-chlorophenyl)-5-hydroxy-1H-pyrazole-4-carboxamide

### EXAMPLE 17A

### 1-(4-Chlorophenyl)-5-hydroxy-1H-pyrazole-4-carboxylic acid

1-(4-Chloro-phenyl)-5-iodo-1H-pyrazole-4-carboxylic acid ethyl ester (0.5 g, 1.3 mmol) (J. Heterocycl. Chem. 1987, 267, 267-270) in EtOH (40 mL) was treated with 20% KOH (10 mL) for 1 h at room temperature. The reaction mixture was concentrated and the residue was partitioned in EtOAc/H₂O. The aqueous layer was acidified to pH 5 and the resulting precipitate was filtered off to give 0.2 g of a (1:3) mixture of 1-(4-chloro-phenyl)-5-iodo-1H-pyrazole-4-carboxylic acid and 1-(4-chloro-phenyl)-5-hydroxy-1H-pyrazole-4-carboxylic acid. MS (ESI+) m/z 238 (M)⁺.

### EXAMPLE 17B

### 1-(4-Chlorophenyl)-5-hydroxy-1H-pyrazole-4-carbonyl chloride

Example 17A (0.06 g, 0.3 mmol) in CH₂Cl₂ (5 mL) was treated with oxalyl chloride (0.1 mL), catalyzed by the addition of a few drops of DMF. After the reaction mixture was stirred at room temperature for 1 h the solvent was evaporated to give the acid chloride.

### EXAMPLE 17C

### N-benzyl-1-(4-chlorophenyl)-5-hydroxy-1H-pyrazole-4-carboxamide

Example 17B was dissolved in THF (3 mL) and reacted with benzylamine (0.50 g, 0.45 mmol) in the presence of triethylamine (0.1 mL) and a catalytic amount of DMAP. The reaction mixture was stirred at ambient temperature for 16 h, the solvents were evaporated in vacuo and the remaining residue was purified by silica gel chromatography (elution with 50% EtOAc/hexandes) to yield the title compound. MS (DCI/NH₃) m/z 329 (M+H)⁺; ¹H NMR (300 MHz, DMSO-d₆) δ 4.47 (d, 2H), 7.30 (m, 5H), 7.60 (d, 2H), 7.88 (d, 2H), 8.20 (s, 1H), 8.76 (t, 1H), 8.98 (s, 1H).

### EXAMPLE 18

### 1-(4-chlorophenyl)-5-hydroxy-N-[3-(methylsulfonyl)phenyl]-1H-pyrazole-4-carboxamide

A solution of Example 17B was treated with 3-(methylsulfonyl)aniline hydrochloride in the presence of triethylamine and a catalytic amount of DMAP as described in Example 17C to yield the title compound. MS (DCI/NH₃) m/z 393 (M+H)⁺; ¹H NMR (DMSO-d₆) δ 3.20 (s, 3H), 7.62 (m, 4H), 7.95 (d, 2H), 8.15 (m, 1H), 8.32 (s, 1H), 8.36 (s, 1H), 9.16 (s, 1H), 11.80 (s, 1H).

### EXAMPLE 19

### 1-(4-chlorophenyl)-5-cyano-N-[3-(methylsulfonyl)phenyl]-1H-pyrazole-4-carboxamide

### EXAMPLE 19A

### 1-(4-Chlorophenyl)-5-cyano-1H-pyrazole-4-carboxylic acid ethyl ester

Ethyl 5-chloro-1-(4-chlorophenyl)-1H-pyrazole-4-carboxylate (J. Heterocycl. Chem. 1987, 267, 267-270) (0.24 g, 0.80 mmol) in acetonitrile (30 mL) was stirred at reflux for 16 h with potassium cyanide (0.12 g, 1.8 mmol) and 18-crown-6 (0.5 g). The solvents were evaporated in vacuo and the residue was chromatographed on silica gel eluting with 2:1 hexane-ether to yield 0.2 g of the title compound. MS (DCI/NH₃) m/z 292 (M+H)⁺; ¹H NMR (CDCl₃) δ 1.40 (t, 3), 4.40 (q, 2H), 7.54 (d, 2H), 7.67 (d, 2H), 8.18 (s, 1H).

### EXAMPLE 19B

### 1-(4-Chlorophenyl)-5-cyano-1H-pyrazole-4-carboxylic acid

Example 19A (0.2 g, 0.7 mmol) was stirred at ambient temperature in EtOH (10 mL) and 20% KOH (5 mL) for 1 h. Ethanol was evaporated in vacuo and the remaining residue was dissolved in water and acidified to yield 0.12 g of the title compound as a tan solid. MS (DCI/NH₃) m/z 265 (M+NH₄)⁺; ¹H NMR (CDCl₃) 7.56 (d, 2H), 7.70 (d, 2H), 8.23 (s, 1H), 12.80 (br s, 1H).

### EXAMPLE 19C

### 1-(4-chlorophenyl)-5-cyano-N-[3-(methylsulfonyl)phenyl]-1H-pyrazole-4-carboxamide

Example 19B (0.1 g, 0.4 mmol) in CH₂Cl₂ was treated with oxalyl chloride (0.1 mL), catalyzed by the addition of DMF. The reaction mixture was stirred at ambient temperature for 2 h, the solvents were removed in vacuo and the obtained acid chloride was dissolved in THF (3 mL) and reacted with 3-methylsulphonylaniline hydrochloride (0.1 g, 0.5 mmol) in the presence of triethylamine (0.14 mL, 1.0 mmol) and a catalytic amount of DMAP. The reaction mixture was stirred at ambient temperature for 16 h, evaporated and purified by chromatography to yield the title compound as a tan solid. MS (DCI/NH₃) m/z 418 (M+NH₄)+; ¹H NMR (DMSO-d₆) δ 3.22 (s, 3H), 7.72 (m, 4H), 7.88 (d, 2H), 8.12 (m, 1H), 8.36 (s, 1H), 8.69 (s, 1H), 9.16 (s, 1H), 10.73 (s, 1H).

### EXAMPLE 20

### 1-(4-chlorophenyl)-N-[3-(methylsulfonyl)phenyl]-5-vinyl-1H-pyrazole-4-carboxamide

### EXAMPLE 20A

### 1-(4-Chlorophenyl)-5-vinyl-1H-pyrazole-4-carboxylic acid ethyl ester

1-(4-Chloro-phenyl)-5-iodo-1H-pyrazole-4-carboxylic acid ethyl ester (0.76 g, 2 mmol) (J. Heterocycl. Chem. 1987, 267, 267-270) was heated for 5 h at reflux in toluene (10 mL) with tributylvinyltin (0.95 g, 3.0 mmol) and dichlorobis[tri(o-tolyl) phosphine]palladium(II) (16 mg). The reaction mixture was evaporated in vacuo and the obtained residue was chromatographed on silica gel (elution with 10% EtOAc/hexanes) to yield 0.60 g of the title compound. MS (DCI/NH₃) m/z 277 (M+NH₄)⁺.

### EXAMPLE 20B

### 1-(4-Chlorophenyl)-5-vinyl-1H-pyrazole-4-carboxylic acid

Example 20A (0.6 g, 2.2 mmol) in EtOH (5 mL) was treated with 20% KOH (2 mL) for 2 h at ambient temperature. Ethanol was evaporated and the aqueous layer was acidified to yield 0.4 g of the title compound as a tan solid. MS (DCI/NH₃) m/z 249 (M+H)⁺; ¹H NMR (DMSO-d₆) δ 5.60 (d, 2H) 6.83 (dd, 1H), 7.52 (d, 2H), 7.62 (d, 2H), 8.02 (s, 1H), 12.63 (br s, 1H).

### EXAMPLE 20C

### 1-(4-Chlorophenyl)-5-vinyl-1H-pyrazole-4-carbonyl chloride

The product from Example 20B (0.4 g, 1.6 mmol) in CH₂Cl₂ (5 mL) was treated with oxalyl chloride (0.5 mL) in the presence of a catalytic amount of DMF. The reaction mixture was stirred at ambient temperature for 2 h upon which the solvent was evaporated to yield the title compound.

### EXAMPLE 20D

### 1-(4-chlorophenyl)-N-[3-(methylsulfonyl)phenyl]-5-vinyl-1H-pyrazole-4-carboxamide

Example 20C (0.054 g, 0.20 mmol) in THF (3 mL) was stirred for 16 h at ambient temperature with 3-methylsulphonylaniline hydrochloride (0.052 g, 0.25 mmol) in the presence of triethylamine (0.7 mL, 0.5 mmol) and a catalytic amount of DMAP. The reaction mixture was evaporated in vacuo and the residue was chromatographed to yield the title compound as a solid. MS (DCI/NH₃) m/z 419 (M+NH₄)⁺; ¹H NMR (DMSO-d₆) δ 3.22 (s, 3H), 5.52 (s, 1H), 5.58 (d, 1H), 6.92 (dd, 1H), 7.55 (d, 2H), 7.65 (m, 4H), 8.05 (m 1H), 8.34 (s, 1H), 8.37 (s, 1H), 10.41 (s, 1H).

### EXAMPLE 21

### 1-(4-chlorophenyl)-N-(3,4-dichlorobenzyl)-5-vinyl-1H-pyrazole-4-carboxamide

The product from the Example 20C (0.054 g, 0.20 mmol) in THF (3 mL) was stirred for 16 h at ambient temperature with 3,4-dichlorobenzylamine (0.044 g, 0.25 mmol) in the presence of triethylamine (0.35 mL, 0.25 mmol) and a catalytic amount of DMAP. The reaction mixture was evaporated in vacuo and the residue was chromatographed to yield the title compound as a solid. MS (DCI/NH₃) m/z 406 (M+H)⁺; ¹H NMR (DMSO-d₆) δ 4.42 (d, 1H), 5.47 (s, 1H), 5.52 (d, 2H), 6.92 (dd, 1H), 7.31 (dd, 1H), 7.5 (d, 2H), 7.62 (m, 4H), 8.18 (s, 1H), 8.82 (t, 1H).

### EXAMPLE 22

### 5-acetyl-1-(4-chlorophenyl)-N-[3-(methylsulfonyl)phenyl]-1H-pyrazole-4-carboxamide

### EXAMPLE 22A

### 5-Acetyl-1-(4-chlorophenyl)-1H-pyrazole-4-carboxylic acid ethyl ester

1-(4-Chlorophenyl)-5-iodo-1H-pyrazole-4-carboxylic acid ethyl ester (0.76 g, 2 mmol) (J. Heterocycl. Chem. 1987, 267, 267-270) was heated for 2 h at reflux in toluene (10 mL) with tributyl(1-ethoxyvinyl)tin (1.0 mL, 3.0 mmol) and dichlorobis[tri(o-tolyl)phosphine]palladium(II) (16 mg). The reaction mixture was evaporated in vacuo and the obtained residue was stirred at ambient temperature for 16 h in 1:1 THF-2N HCl (5 mL). The reaction mixture was evaporated and the obtained residue was chromatographed on silica gel (elution with 10% EtOAc/hexanes) to yield 0.20 g of the title compound. MS (DCI/NH₃) m/Z 293 (M+H)⁺; ¹H NMR (DMSO-d₆) δ 1.28 (t, 3H), 2.67 (s, 3H), 4.28 (q, 2H), 7.50 (d, 2H), 7.61 (d, 2H), 8.16 (s, 1H).

### EXAMPLE 22B

### 5-Acetyl-1-(4-chlorophenyl)-1H-pyrazole-4-carboxylic acid

Example 22A (0.2 g, 0.7 mmol) in EtOH (5 mL) was treated with 20% KOH (2 mL) for 2 h at ambient temperature. Ethanol was evaporated and the aqueous layer was acidified to yield 0.14 g of the title compound as a tan solid. MS (DCI/NH₃) m/z 266(M+H)⁺; ¹H NMR (DMSO-d₆) δ 2.67 (s, 3H), 7.50 (d, 2H), 7.60 (d, 2H), 8.10 (s, 1 h), 12.80 (br s, 1H).

### EXAMPLE 22C

### 5-Acetyl-1-(4-chlorophenyl)-1H-pyrazole-4-carbonyl chloride

Example 22B (0.2 g, 0.8 mmol) in CH₂Cl₂ (5 mL) was treated with oxalyl chloride (0.4 mL) in the presence of a catalytic amount of DMF. The reaction mixture was stirred at ambient temperature for 2 h upon which the solvent was evaporated to yield the tiltle compound.

### EXAMPLE 22D

### 5-acetyl-1-(4-chlorophenyl)-N-[3-(methylsulfonyl)phenyl]-1H-pyrazole-4-carboxamide

Example 22C (0.048 g, 0.17 mmol) in THF (3 mL) was stirred for 16 h at ambient temperature with 3-(methylsulfonyl)aniline hydrochloride (0.043 g, 0.21 mmol) in the presence of triethylamine (0.7 mL, 0.5 mmol) and a catalytic amount of DMAP. The reaction mixture was evaporated in vacuo and the residue was chromatographed to yield the title compound as a solid. MS (DCI/NH₃) m/z 435 (M+NH₄)⁺; ¹H NMR (DMSO-d₆) δ 2.61 (s, 3H), 3.22 (s, 3H), 7.51 (d, 2H), 7.61 (d, 2H), 7.66 (d, 2H), 8.08 (s, 1H), 8.33 (s, 1H), 8.39 (s, 1H), 10.7 (s, 1H).

### EXAMPLE 23

### 5-acetyl-1-(4-chlorophenyl)-N-(3,4-dichlorobenzyl)-1H-pyrazole-4-carboxamide

Example 22C (0.048 g, 0.17 mmol) in THF (3 mL) was stirred for 16 h at ambient temperature with 3,4-dichlorobenzylamine (0.035 g, 0.20 mmol) in the presence of triethylamine (0.35 mL, 0.25 mmol) and a catalytic amount of DMAP. The reaction mixture was evaporated in vacuo and the residue was chromatographed to yield the title compound as a solid. MS (DCI/NH₃) m/z 423 (M+H)⁺; ¹H NMR (DMSO-d₆) δ 2.6 (s, 3H), 4.43 (d, 2H), 7.32 (dd, 1H), 7.48 (d, 2H), 7.58 (d, 2H), 7.62 (d, 2H), 8.2 (s, 1H), 9.12 (t, 1H).

### EXAMPLE 24

### 1-(4-chlorophenyl)-N-(2-methoxybenzyl)-5-methyl-1H-pyrazole-3-carboxamide

A solution of the product from Example 14D and 2-methoxybenzylamine were processed as described in Example 1D to provide the desired product. MS (ESI) m/z 356 (M+H)⁺; ¹H NMR (300 MHz, DMSO-d₆) δ 8.48 (t, 1H, J=6.1 Hz), 7.60-7.69 (m, 4H), 7.22 (ddd, 1H, J=8.1, 7.5, 1.7 Hz), 7.14 (dd, 1H, J=7.5, 1.7 Hz), 6.98 (dd, 1H, J=8.5, 1.0 Hz), 6.89 (t, 1H, J=7.5, 1.0 Hz), 6.68 (d, 1H, J=0.7 Hz), 4.40 (d, 2H, J=6.1 Hz), 3.82 (s, 3H), 2.35 (s, 3H).

### EXAMPLE 25

### 1-(4-chlorophenyl)-5-methyl-N-(2-methylbenzyl)-1H-pyrazole-3-carboxamide

A solution of the product from Example 14D and 2-methylbenzylamine were processed as described in Example 1D to provide the desired product. MS (ESI) m/z 340 (M+H)⁺; ¹H NMR (300 MHz, DMSO-d₆) δ 8.60 (t, 1H, J=6.1 Hz), 7.96-7.59 (m, 4H), 7.26-7.19 (m, 2H), 7.17-7.10 (m, 2H), 6.68 (d, 1H, J=0.7 Hz), 4.41 (d, 2H, J=6.1 Hz), 2.35 (s, 3H), 2.31 (s, 3H).

### Functional Studies on Tetrodotoxin Resistant (TTX-R) Currents in Neuropathic Pain Model

Abnormal activity of sodium channels in the peripheral nervous system plays a role in the pathophysiology of chronic pain. Sodium channels are critical elements in the transduction of action potentials in excitable tissues such as nerve and muscle, and as such, participate in many physiological processes. The recent identification of sensory neuron-specific sodium channels such as Nav1.8, the observation that their expression is altered in chronic pain states, and the demonstration that Nav1.8 antisense attenuates pain in animal models suggest that these channels are attractive targets for drug discovery. Given the restricted expression pattern of these channels, selective blockers could, in principle, be effective analgesics without undesirable effects observed with nonselective sodium channel blockers. Although excitability of sensory neurons can be modulated by various receptors and ion channel processes, sodium channels directly regulate neuronal excitability.

Spontaneously ectopic action potential firing in dorsal root ganglion (DRG) neurons is believed to be the underlying mechanism that evokes neuropathic pain following nerve injury. It has been recognized for some time that tetrodotoxin-resistant (TTX-R) current increases in chronic pain, and several studies have implicated Nav1.8 as the primary channel responsible for this increased current.

To examine functional effects, TTX-R sodium currents were studied in dorsal root ganglion (DRG) neurons from rats 14 days following spinal nerve ligation (SNL). Small size neurons (C-fiber neurons <25 μm) from L4 and L5 DRG were dissociated from rats, and Na⁺ currents were measured in the presence of 100 nM TTX by whole-cell current recording. The total Na⁺ current density was reduced by 42% in L5 DRG neurons from SNL rats compared to L5 DRG neurons from sham operated rats. This reduction in current density was attributable to a significant reduction in TTX-resistant, but not TTX-sensitive currents. Moreover, this decrease in TTX-R current was observed only in the L5 injured region, whereas there was a significant increase in TTX-R currents in the L4 uninjured region.

Although TTX-R currents were significantly decreased in L5 ganglia, the increased TTX-R currents in L4 ganglia may be responsible for the firing and sensation of neuropathic pain. A Nav1.8 channel inhibitor may attenuate neuropathic pain by blocking currents in L4 DRG neurons, as well as by blocking currents generated at the nociceptive peripheral terminals. Representative compounds of the present invention demonstrated IC₅₀ₛ from about 500 nM to about 3 μM.

Compounds of the present invention inhibit the PN3 sodium channel and are therefore useful as analgesics for neuropathic pain.

The present invention also provides pharmaceutical compositions that comprise compounds of the present invention. The pharmaceutical compositions comprise compounds of the present invention formulated together with one or more non-toxic pharmaceutically acceptable carriers.

The pharmaceutical compositions of this invention can be administered to humans and other mammals orally, rectally, parenterally, intracisternally, intravaginally, topically (as by powders, ointments or drops), bucally or as an oral or nasal spray. The term “parenterally,” as used herein, refers to modes of administration which include intravenous, intramuscular, intraperitoneal, intrasternal, subcutaneous and intraarticular injection and infusion.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable carrier or excipient, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of this invention with suitable non-irritating carriers or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration of a compound of this invention include powders, sprays, ointments and inhalants. The active compound may be mixed under sterile conditions with a pharmaceutically acceptable carrier and any needed preservatives, buffers or propellants which may be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated.

When used in the above or other treatments, a therapeutically effective amount of one of the compounds of the present invention can be employed in pure form or, where such forms exist, in pharmaceutically acceptable salt, ester or prodrug form. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The term “pharmaceutically acceptable salt,” as used herein, means salts derived from inorganic or organic acids. The salts can be prepared in situ during the final isolation and purification of compounds of the present invention or separately by reacting the free base of a compound of a compound of the present invention with an inorganic or organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, dihydrochloride, hydrobromide, hydroiodide, 2-hydroxyethansulfonate (isethionate), lactate, maleate, fumarate, methanesulfonate, nicotinate, 2-naphthalenesulfonate, oxalate, pamoate, pectinate, persulfate, 3-phenylpropionate, picrate, pivalate, propionate, succinate, sulfate, (L) tartrate, (D) tartrate, (DL) tartrate, thiocyanate, phosphate, glutamate, bicarbonate, p-toluenesulfonate, and undecanoate. Representative examples include, but are not limited to N-(1-benzylpiperidin-4-yl)-5-methyl-1-phenyl-1H-pyrazole-4-carboxamide hydrochloride, N-(1-benzylpiperidin-4-yl)-5-methyl-1-phenyl-1H-pyrazole-4-carboxamide (L) tartrate, or N-(1-benzylpiperidin-4-yl)-5-methyl-1-phenyl-1H-pyrazole-4-carboxamide benzene sulfonate.

The term “pharmaceutically acceptable ester,” as used herein, means esters of compounds of the present invention which hydrolyze in vivo and include those that break down readily in the human body to leave the parent compound or a salt thereof. Examples of pharmaceutically acceptable, non-toxic esters of the present invention include C₁-to-C₆ alkyl esters and C₅-to-C₇ cycloalkyl esters, although C₁-to-C₄ alkyl esters are preferred. Esters of the compounds of the present invention may be prepared according to conventional methods. Representative examples include, but are not limited to, ethyl 4-(4-{[(1-benzylpiperidin-4-yl)amino]carbonyl}-5-methyl-1H-pyrazol-1-yl)benzoate or methyl 4-(4-{[(1-benzylpiperidin-4-yl)amino]carbonyl}-5-methyl-1H-pyrazol-1-yl)benzoate.

The term “pharmaceutically acceptable amide,” as used herein, means to non-toxic amides of the present invention derived from ammonia, primary C₁-to-C₆ alkyl amines and secondary C₁-to-C₆ dialkyl amines. In the case of secondary amines, the amine may also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of the present invention may be prepared according to conventional methods. Representative examples include, but are not limited to, 1-[4-(aminocarbonyl)phenyl]-N-(1-benzylpiperidin-4-yl)-5-methyl-1H-pyrazole-4-carboxamide or N-(1-benzylpiperidin-4-yl)-1-{4-[(dimethylamino)carbonyl]phenyl}-5-methyl-1H-pyrazole-4-carboxamide.

The term “pharmaceutically acceptable prodrug” or “prodrug,” as used herein, represents those prodrugs of the compounds of the present invention which are, within the scope of sound medical judgement, suitable for use in contact with the tissues of humans and lower animals without undue toxicity, irritation, allergic response, and the like. Prodrugs of the present invention may be rapidly transformed in vivo to compounds of the present invention, for example, by hydrolysis in blood. Representative examples include, but are not limited to, N-acetyl-N-(1-benzylpiperidin-4-yl)-5-methyl-1-phenyl-1H-pyrazole-4-carboxamide, N-(1-benzylpiperidin-4-yl)-N-(2,2-dimethylpropanoyl)-5-methyl-1-phenyl-1H-pyrazole-4-carboxamide, or ethyl 1-benzylpiperidin-4-yl[(5-methyl-1-phenyl-1H-pyrazol-4-yl)carbonyl]carbamate.

The present invention contemplates compounds of the present invention formed by synthetic means or formed by in vivo biotransformation.

The compounds of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The total daily dose of the compounds of this invention administered to a human or lower animal may range from about 0.01 to about 125 mg/kg/day. For purposes of oral administration, more preferable doses can be in the range of from about 0.1 to about 150 mg/kg/day. If desired, the effective daily dose can be divided into multiple doses for purposes of administration; consequently, single dose compositions may contain such amounts or submultiples thereof to make up the daily dose.

1. A compound of formula (I) 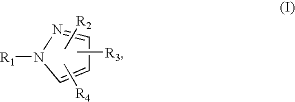
or a pharmaceutically acceptable salt, amide, ester, or prodrug thereof, wherein
R₁ is alkyl, aryl, arylalkyl, cycloalkyl, cycloalkylalkyl, haloalkyl, heterocycle, heterocyclealkyl, heteroaryl, or heteroarylalkyl;
R₂ and R₃ are independently hydrogen, alkenyl, alkoxy, alkoxyalkyl, alkoxycarbonyl, alkyl, alkylcarbonyl, alkylcarbonyloxy, alkylthio, alkynyl, aryl, arylalkyl, carboxy, cycloalkyl, cycloalkylalkyl, cyano, formyl, haloalkoxy, haloalkyl, halogen, hydroxy, hydroxyalkyl, mercapto, nitro, —NR_{A}R_{B}, or (NR_{A}R_{B})carbonyl;
R_{A} and R_{B} are independently hydrogen, alkyl, or alkylcarbonyl;
R₄ is 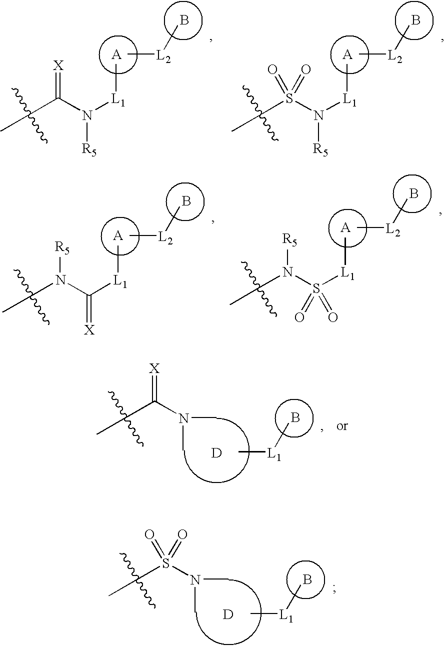
X is O or S;
R₅ is hydrogen, alkyl, alkylcarbonyl, alkylcarbonyloxy, or heterocyclealkyl;
L₁ is a bond or alkylene;
L₂ is a bond or alkylene;
A is aryl, cycloalkyl, heteroaryl, or heterocycle;
B is aryl, cycloalkyl, heteroaryl, or heterocycle;
D is heterocycle wherein the heterocycle is azetidinyl, azepanyl, aziridinyl, azocanyl, 1,1-dioxidothiomorpholinyl, morpholinyl, piperazinyl, piperidinyl, pyrrolidinyl, or thiomorpholinyl, wherein the heterocycle is optionally substituted with 1, 2, 3, or 4 substitutents independently selected from alkenyl, alkoxy, alkoxyalkyl, alkoxycarbonyl, alkoxysulfonyl, alkyl, alkylcarbonyl, alkylcarbonyloxy, alkylsulfonyl, alkynyl, carboxy, cyano, formyl, haloalkoxy, haloalkyl, halo, hydroxy, hydroxyalkyl, mercapto, —NR_{A}R_{B}, (NR_{A}R_{B})carbonyl, or (NR_{A}R_{B})sulfonyl.

2. The compound according to claim 1 wherein
R₁ is aryl; and
R₄ is

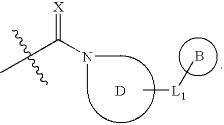

3. The claim according to claim 1 wherein
R₁ is aryl;
R₄ is 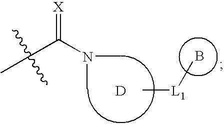
X is O;
D is piperazinyl;
L₁ is a bond; and
B is aryl.

4. The claim according to claim 1 wherein
R₁ is aryl wherein the aryl is phenyl substituted with 1 halogen substituent;
R₂ is hydrogen;
R₃ is haloalkyl;
R₄ is 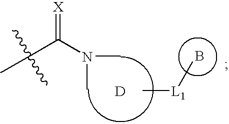
X is O;
D is piperazinyl;
L₁ is a bond; and
B is aryl wherein the aryl is phenyl substituted with 1 halogen substituent.

5. The compound according to claim 4 that is 1-(3-chlorophenyl)-4-{[1-(4-chlorophenyl)-5-(trifluoromethyl)-1H-pyrazol-4-yl]carbonyl} piperazine.

6. The claim according to claim 1 wherein
R₁ is aryl wherein the aryl is phenyl substituted with 1 halogen substituent;
R₂ is hydrogen;
R₃ is haloalkyl;
R₄ is 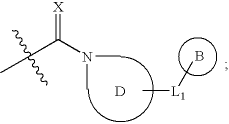
X is O;
D is piperazinyl;
L₁ is a bond; and
B is cycloalkyl wherein the cycloalkyl is cyclohexyl.

7. The compound according to claim 4 that is 1-{[1-(4-chlorophenyl)-5-(trifluoromethyl)-1H-pyrazol-4-yl]carbonyl}-4-cyclohexylpiperazine.

8. The compound according to claim 1 wherein
R₁ is aryl; and
R₄ is

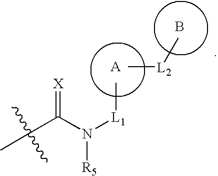

9. The claim according to claim 1 wherein
R₁ is aryl;
R₄ is 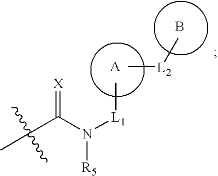
R₅;
X is O;
L₁ is a bond;
A is piperidinyl;
L₂ is alkylene; and
B is aryl.

10. The claim according to claim 1 wherein
R₁ is aryl wherein the aryl is phenyl substituted with 1 halogen substituent;
R₂ is hydrogen;
R₃ is alkyl;
R₄ is 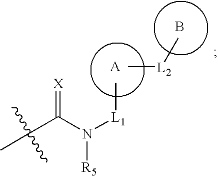
X is O;
L₁ is a bond;
A is piperidinyl;
L₂ is alkylene; and
B is aryl wherein the aryl is phenyl.

11. The compound according to claim 4 that is N-(1-benzylpiperidin-4-yl)-1-(4-chlorophenyl)-5-methyl-1H-pyrazole-4-carboxamide.

12. A compound of formula (II) 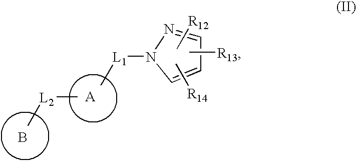
or a pharmaceutically acceptable salt, amide, ester, or prodrug thereof, wherein
R₁₂ and R₁₃ are independently hydrogen, alkenyl, alkoxy, alkoxyalkyl, alkoxycarbonyl, alkyl, alkylcarbonyl, alkylcarbonyloxy, alkylthio, alkynyl, aryl, arylalkyl, carboxy, cycloalkyl, cycloalkylalkyl, cyano, formyl, haloalkoxy, haloalkyl, halogen, hydroxy, hydroxyalkyl, mercapto, nitro, —NR_{A}R_{B}, or (NR_{A}R_{B})carbonyl;
R_{A} and R_{B} are independently hydrogen, alkyl, or alkylcarbonyl;
R₁₄ is 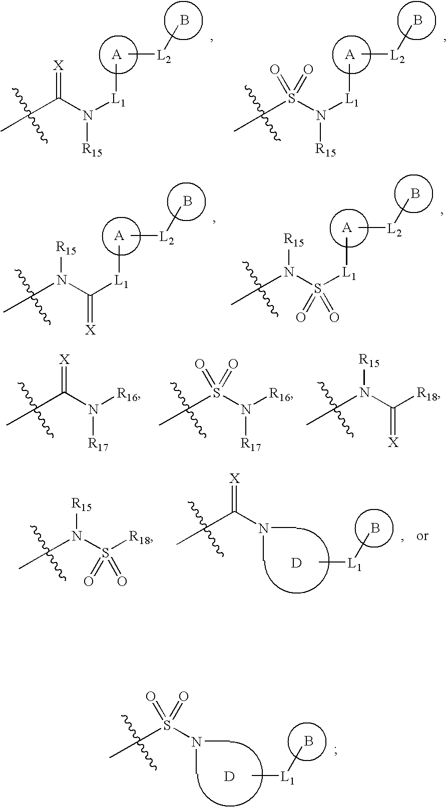
X is O or S;
R₁₅ is hydrogen or alkyl;
R₁₆ and R₁₇ are independently hydrogen, alkenyl, alkoxy, alkyl, aryl, arylalkyl, cycloalkyl, cycloalkylalkyl, heteroaryl, heteroarylalkyl, heterocycle, or heterocyclealkyl;
R₁₈ is alkyl, alkenyl, aryl, arylalkyl, cycloalkyl, cycloalkylalkyl, heteroaryl, heteroarylalkyl, heterocycle, heterocyclealkyl, —NR_{A}R_{B}, or (NR_{A}R_{B})alkyl;
L₁ is a bond or alkylene;
L₂ is absent, a bond, or alkylene;
A is aryl, cycloalkyl, heteroaryl, or heterocycle;
B is absent, aryl, cycloalkyl, heteroaryl, or heterocycle;
D is heterocycle wherein the heterocycle is azetidinyl, azepanyl, aziridinyl, azocanyl, 1,1-dioxidothiomorpholinyl, morpholinyl, piperazinyl, piperidinyl, pyrrolidinyl, or thiomorpholinyl, wherein the heterocycle is optionally substituted with 1, 2, 3, or 4 substitutents independently selected from alkenyl, alkoxy, alkoxyalkyl, alkoxycarbonyl, alkoxysulfonyl, alkyl, alkylcarbonyl, alkylcarbonyloxy, alkylsulfonyl, alkynyl, carboxy, cyano, formyl, haloalkoxy, haloalkyl, halo, hydroxy, hydroxyalkyl, mercapto, —NR_{A}R_{B}, (NR_{A}R_{B})carbonyl, or (NR_{A}R_{B})sulfonyl.

13. The compound according to claim 12 wherein
L₁ is a bond;
A is heterocycle;
L₂ is alkylene;
B is aryl; and
R₁₄ is

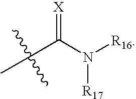

14. The compound according to claim 12 wherein
L₁ is a bond;
A is heterocycle wherein the heterocycle is piperidinyl;
L₂ is alkylene;
B is aryl wherein the aryl is phenyl;
R₁₄ is 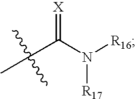
X is O;
R₁₂ and R₁₆ are hydrogen;
R₁₃ is haloalkyl; and
R₁₇ is aryl wherein the aryl is phenyl substituted with 1 alkylsulfonyl substituent.

15. The compound according to claim 14 that is 1-(1-benzylpiperidin-4-yl)-N-[3-(methylsulfonyl)phenyl]-5-(trifluoromethyl)-1H-pyrazole-4-carboxamide.

16. The compound according to claim 12 wherein
L₁ is a bond;
A is heterocycle;
L₂ and B are absent; and
R₁₄ is

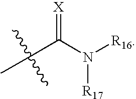

17. The compound according to claim 12 wherein
L₁ is a bond;
A is heterocycle wherein the heterocycle is tetrahydropyran;
L₂ and B are absent; and
R₁₄ is

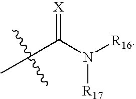

18. A compound that is
1-(3-chlorophenyl)-N-[3-(methylsulfonyl)phenyl]-5-(trifluoromethyl)-1H-pyrazole-4-carboxamide;
1-(3-methylphenyl)-N-[3-(methylsulfonyl)phenyl]-5-(trifluoromethyl)-1H-pyrazole-4-carboxamide;
1-(4-methylphenyl)-N-[3-(methylsulfonyl)phenyl]-5-(trifluoromethyl)-1H-pyrazole-4-carboxamide;
1-(2-methoxyphenyl)-5-(trifluoromethyl)-N-[3-(trifluoromethyl)benzyl]-1H-pyrazole-4-carboxamide;
N-[2-(4-chlorophenyl)ethyl]-1-(2-methoxyphenyl)-5-(trifluoromethyl)-1H-pyrazole-4-carboxamide;
1-cyclohexyl-5-(trifluoromethyl)-N-[3-(trifluoromethyl)benzyl]-1H-pyrazole-4-carboxamide;
1-cyclohexyl-N-[3-(methylsulfonyl)phenyl]-5-(trifluoromethyl)-1H-pyrazole-4-carboxamide;
N-[2-(4-chlorophenyl)ethyl]-1-(7-chloroquinolin-4-yl)-5-(trifluoromethyl)-1H-pyrazole-4-carboxamide;
1-(4-chlorophenyl)-5-methyl-N-[3-(trifluoromethyl)benzyl]-1H-pyrazole-4-carboxamide;
1-(4-chlorophenyl)-N-[2-(4-chlorophenyl)ethyl]-5-methyl-1H-pyrazole-3-carboxamide;
1-(4-chlorophenyl)-5-methyl-N-[3-(trifluoromethyl)benzyl]-1H-pyrazole-3-carboxamide;
1-(4-chlorophenyl)-5-methyl-N-[3-(methylsulfonyl)phenyl]-1H-pyrazole-3-carboxamide;
N-benzyl-1-(4-chlorophenyl)-5-hydroxy-1H-pyrazole-4-carboxamide;
1-(4-chlorophenyl)-5-hydroxy-N-[3-(methylsulfonyl)phenyl]-1H-pyrazole-4-carboxamide;
1-(4-chlorophenyl)-5-cyano-N-[3-(methylsulfonyl)phenyl]-1H-pyrazole-4-carboxamide;
1-(4-chlorophenyl)-N-[3-(methylsulfonyl)phenyl]-5-vinyl-1H-pyrazole-4-carboxamide;
1-(4-chlorophenyl)-N-(3,4-dichlorobenzyl)-5-vinyl-1H-pyrazole-4-carboxamide;
5-acetyl-1-(4-chlorophenyl)-N-[3-(methylsulfonyl)phenyl]-1H-pyrazole-4-carboxamide;
5-acetyl-1-(4-chlorophenyl)-N-(3,4-dichlorobenzyl)-1H-pyrazole-4-carboxamide;
1-(4-chlorophenyl)-N-(2-methoxybenzyl)-5-methyl-1H-pyrazole-3-carboxamide; or
1-(4-chlorophenyl)-5-methyl-N-(2-methylbenzyl)-1H-pyrazole-3-carboxamide; or a pharmaceutically acceptable salt, amide, ester, or prodrug thereof.
